(19) United States
(12) Patent Application Publication
(10) Pub. No.: US 2025/0320511 A1
(43) Pub. Date: Oct. 16, 2025

(54) CHROMOSOMAL INTEGRATING CASSETTE ALLOWING INDUCIBLE GENE EXPRESSION FOR PRODUCTION OF COMPOUNDS VIA FERMENTATION

(71) Applicant: Justus-Liebig-Universität Giessen, Giessen (DE)

(72) Inventors: Matthew MCINTOSH, Giessen (DE); Gabriele KLUG, Pohlheim (DE); Jonas KRETZ, Giessen (DE)

(21) Appl. No.: 18/684,250

(22) PCT Filed: Aug. 17, 2022
(86) PCT No.: PCT/EP2022/072999

§ 371 Date: Feb. 16, 2024

(30) Foreign Application Priority Data
Aug. 17, 2021 (EP) 21191750.5

(51) Int. Cl. C12N 15/72; C12N 1/20; C12N 15/67; C12N 15/74; C12N 15/90

(52) U.S. Cl. CPC C12N 15/72; C12N 1/20; C12N 15/67; C12N 15/74; C12N 15/902

(57) ABSTRACT

The present invention provides a new system, resp. method, of genetic modification, called ACIT (Alphaproteobacteria chromosomally inserting transcription-control cassette). The invention, however, is not limited to use in Alphaproteobacteria. The modification can be implemented within the bacterial chromosome and therefore permanently implemented within the microorganism.

Microbial fermentation processes are important methods for producing chemical compounds in a sustainable way, because their feedstock consist of agriculture-based raw materials and they are usually less energy-consuming than classical processes due to the low fermentation temperatures in comparison with classical chemical reactions.

### FIELD OF THE INVENTION

The invention relates to the field of production by microbial fermentation of substances or mixtures of substances, preferably fine chemicals, e.g. curdlan, polyhydroxybutyrate (PHB) or galactoglucan, by use of a novel inducible gene expression system. The invention is designed to modify bacterial chromosomes in order to achieve tight control over inducible gene expression (see FIG. 1).

### BACKGROUND OF THE TECHNOLOGY

Regulation of gene expression is essential for survival. The mere presence of genes (i.e., the genome) does not ensure survival. Rather, each gene needs to be expressed appropriately, at the right rate and the right timing, according to its role in the survival of the organism. Many, if not most, of the regulatory mechanisms that control gene expression throughout nature remain undiscovered. Manipulation of gene expression holds immense potential for taking advantage of the impressive abilities of life. For example, modified bacterial pathogens could be used in medicine to kill genetically related bacteria through the control of gene expression. Artificial control over gene expression is also useful in bacterial cell factories for the production of antibiotics and useful vitamins, proteins and polysaccharides and renewable energy sources. Manipulation of gene expression is a favourite strategy in biology for the study of gene regulatory circuitry necessary for life. It is useful for detecting and characterizing novel regulatory mechanisms. Current state of the art research offers a number of possibilities for manipulating gene expression, using a range of physical and chemical inducers, each involving advantages and disadvantages. However, the problems involved with achieving stable and controllable gene expression are many.

Inducible gene expression is currently (within the current state of the art) applied using inducible expression systems carried on plasmids. A plasmid is an extrachromosomal DNA molecule within a cell that can replicate independently of the chromosome. There are at least four major problems with inducing gene expression using plasmids. Firstly, plasmids can be lost during cell reproduction, particularly when they create a burden on cellular metabolism. Prevention of plasmid loss can be minimized by the use of selective criteria, such as the inclusion of antibiotics in the growth medium. However, these tend to have diverse negative side effects on cell growth and other bacterial behaviours. Secondly, most replicating plasmids exist at 5-100 copies per cell, which is suitable for inducible overexpression but not repression of expression. Multiple copies of the inducible expression system mean that leaky or unwanted expression will always be present. Thirdly, whenever the inducible expression of several genes that are organized in an operon (an assembly of co-transcribed genes) is desired, this requires the laborious cloning of these genes into a plasmid, a task that can consume considerable time and resources. Fourthly, a plasmid is unable to control the expression of the genes of interest while located at their natural site in the chromosome. The most common solution to this problem to date is to first delete the gene of interest from the bacterial chromosome (to create a mutant), and then to reintroduce the gene of interest under the control of an inducible gene expression system carried on a plasmid. However, this solution does not avoid the leaky expression problem associated with multiple copy numbers of the plasmid. It also requires considerable efforts to generate the mutant as well as cloning the gene of interest. Furthermore, it creates an unstable genome (i.e., upon addition of an extra-chromosomal plasmid) that must be maintained using antibiotics.

Upon induction, an inducible gene expression system should provide the desired level of gene expression. Typically a strong gene expression is favourable. In many systems known within the state of the art, the induced expression is simply too weak to ensure significant biosynthesis of enzymes, pharmaceuticals, biofuels and other valuable polymers or other fine chemicals. This is especially problematic when transferring well-characterized inducible expression systems from E. coli into other industrially relevant bacteria, such as the Alphaproteobacterium Rhodobacter sphaeroides. These systems in R. sphaeroides appeared weak, leaky, or both, for unknown reasons. Weak expression, for example, could be due to incompatibility between the inducible promoter and the RNA polymerase of the host organism. See, for example: T. Shi et al. (2021) “Screening and engineering of high-activity promoter elements through transcriptomics and red fluorescent protein visualization in Rhodobacter sphaeroides”, Synthetic and Systems Biotechnology 6:335-342 and A. O. Johnson et al. (2018) “An Engineered Constitutive Promoter Set with Broad Activity Range for Cupriavidus necator H16”, ACS Synth Biol 7:1918-1928.

Document US20170002363A1 reveals a system for genetic modification including the application of the EiL-System for modulating the degree of genetic expression in a broad range of bacteria. The Eil system consists of the eilR gene and the promoter controlled by the EilR protein that activates expression of a gene of interest, for example the lacI gene. The EiL-System ensures the control over the expression of a gene of interest, for example the lacI-system by usage of crystal violet as the inducer for the repressive effect of the EilR protein and therefore indirectly as the inhibitor of gene expression. The system described in US20170002363A1 cannot be implemented chromosomically, i.e. within chromosomes; it is confined to be implemented within separate plasmid-structures, to be inserted additionally into the microbial cells.

The current state of the art, i.e. inserting the desired genetic modification to the microbial cells via additional plasmid-structures is prone to a gradual loss of the genetic information, so that “refreshing” is necessary after several generations of microbial growth.

### CONTENT OF THE INVENTION

The objective of this invention is two-fold: the design of a genetic system capable of tight control of expression of one or several genes of interest, and the design of a genetic system capable of being adjusted and adapted to enhance operation in any bacterium using naturally occurring or modified versions of any promoter of interest.

In order to achieve the above objective, the present invention provides a new system, resp. method, of genetic modification, called hereinafter ACIT (Alphaproteobacteria suitable chromosomally inserting transcription-control cassette). The invention, however, is not limited to use in Alphaproteobacteria. ACIT is operatively linked to the gene of interest as is described below, allowing the control of the gene of interest.

ACIT solves the problems described above by using a plasmid that integrates into the host chromosome of the bacterial host. By such stable integration the later loss of plasmids during cell reproduction is avoided; the possibility is provided to implement not only overexpression, but also repression of expression in order to fine-tune the expression; if inducible expression of several genes that are organized in an operon is desired, it is no longer necessary to perform the laborious cloning of these genes into a plasmid; controlling the expression of the genes of interest while being located at their natural sites in the chromosome is possible.

The integration into the chromosome of the bacterial host is specific, targeting the native site of the gene of interest via homologous recombination (see FIG. 2). There are several striking advantages of using an integrated plasmid. Firstly, plasmids integrated into the chromosomes are more stable than self-replicating plasmids, since these are far less likely to be lost than replicating plasmids, particularly when they create a burden on cellular metabolism. Hence, antibiotics are not necessary for their maintenance. Secondly, the use of an integrating plasmid in ACIT provides for a tighter control over gene expression. While most replicating plasmids exist at 5-100 copies per cell, integrated plasmids adopt the copy number of a chromosome, i.e., 1-2 copies per cell. This allows the controlled transcription depletion or silencing of the gene of interest without disruption of gene sequence. Thus, a tremendous advantage of ACIT is the control over a single copy of the gene of interest. Thirdly, ACIT can control the expression of an operon of genes in their native location on the chromosome without altering their physical location on the chromosome and without the need to clone the entire operon into an extrachromosomal plasmid.

ACIT solves further major problems in the field of inducible expression, e.g.: Within microbiology, most of the information on available inducer system comes from the gammaproteobacteria, such as Escherichia coli, or from Streptomyces such as Bacillus subtilis. Less is known about inducible systems outside of these model bacteria. Moreover, application of inducible systems from model bacteria in lesser-studied bacteria is fraught with problems. For example, the alphaproteobacterium Sinorhizobium meliloti is an agriculturally important soil bacterium because of its ability to fix atmospheric nitrogen in symbiotic association with alfalfa (Medicago sativa). An arabinose-inducible system, which works well in E. coli, does not function in S. meliloti for unknown reasons. Thus, the arabinose-inducible system cannot be used in S. meliloti. This example underscores the importance of developing inducible expression systems that are useful in a broad range of bacteria. ACIT is addressing this need and solves this problem since its design allows for adjustment and modifying it to make it suitable for use in any bacterium, including both model and non-model bacteria as well as less-studied model bacteria.

The method of genetic modification via ACIT is exemplarily described by a process for controlling cell growth and cell behavior and for producing exopolysaccharides and other compounds of interest.

The invention consists of six specifically arranged nucleotide sequences, referred to in this document as parts (for example synonymously as part 1, resp. part i), or as part 2, resp. part ii) etc.). Specific to this invention is (a) the order of the six parts, which is essential for the functioning of ACIT and (b) the nucleotide sequence of part one. The benefit of ACIT is to achieve an extremely efficient control over the transcription rate of a chromosomally located gene of interest through the addition of chemical inducers. Isopropyl-R-D-thiogalactopyranoside (IPTG) is applied here for fine-tuning the transcription activation. Upon making the appropriate adjustments to ACIT, other chemicals such as crystal violet may serve as the chemical inducer of tight transcription control.

Part one is a promoter sequence flanked by repressor binding sites, preferably Lac operator sites. This nucleotide sequence is novel, since it is a combination of any promoter of interest flanked by Lac operator sites, and is the most important part in this invention, since it provides the ability to control the expression of a specifically chosen gene of interest using compounds of initiation for example but not limited to allolactose, IPTG or other galactose thioglycosides as chemical inducers of gene expression. Examples of lac operator sequences are provided as SEQ ID no. 1 and SEQ ID no. 2.

Part two is a nucleotide sequence for a repressor of gene expression that represses the promoter of part one via the repressor binding sites, for example a lacI gene coding for a LacI repressor protein suitable for the lac operator sites of part one. Examples of lacI genes are given as SEQ ID no 8 and the following SEQ ID no. 33.

>NC_020528.1: c757440-756370 Sinorhizobium
meliloti 2011, complete sequence:
(SEQ ID no. 33)
ATGCCGGTCAATCTCAAGCAATTGGCGGAACTGCTCGGCCTGTCCCAGA
CCACGGTCAGCCGGGCGCTGAACGGCTATCCGGAGGTCAATGCGGAAAC
GCGGGCACGGGTGCTGGAGGCGGTGCGCGAGACGGGATACAGGCCCAAC
CGTGCCGCACAGCGCCTCGCGACCGGGAAAGCCTATTCGATCGGGCTCG
TCATGCCGATCGCAGCCGGAATCGATTCGGACATCCATTTCGGTGAATT
CCTGGCGGGGCTGGCGGAAGAAGCGGTGGAGCACGATTTCCACTTCGTG
TTGAACCCCAGTGCGCCCGAAGACGAGGAGGCGACATTCCGCCGGCTTG
CAGCGAGCGGCAATGTCGACGCTGTCTTCATCGCCTATATGCGCGCCAA
CGATCCCCGCATCGAGATGCTGAAGGCACTTTCCATTCCCTTCGTGGTG
CATGGCCGCTCCATCGGCGGTCCCCGGGACTATCCGTTCGTCGACGTGG
ACAATACCGGCGCCTTCTACGACGCCGCCAGGCTTCTCATCCAGCTCGG
CCACAACCGCATCGCCCTGATCAACGGGCCGGAACACCTCACCTTCTCC
ATCCGCAGGAGGAAGGGGCTGGTGCGCGCGCTCGCGGAGAAGGGTCTCA
ACCTCGACGATGCGCTCGTCCATCACTCTGCGATGACCGACGAGCATGG
CTATCGGAGCATGCAGCGTTTCCTCAAACGGCCGGCCCCGCCGACCGCC
GTCCTTTGTTCCAGTACAGTGCTGGCGCTCGGCGCCGTACGGGCGATCA
ACCAGGCCGGACTCGCCATCGGCACCGACATCTCGCTCATCGCCCATGA
CGACGTGCTGCCGATGCTGAAGCCCGAGAACTTCAGCGTACCGCTGACG
ACGACGCGCTCCTCGCTTCGCGCGGCAGGTGCACGCATCGCCAAGCGGC
TGATCGGCGGCATACTCAATCAGGGAGACTATCCCGAGCAGGAGCTTTG
GCGCGCCGAACTGATCGTGCGTGCCTCGACGGGGCCTGCACCGGACAGA
TCACCCCTCCCCAACCCCTCCCCACAGGTGGGAGGGGCTTAA

An example of a LacI repressor protein suitable for the Lac operator sites of part one is the following protein sequence SEQ ID no. 32, originating from Sinorhizobium.

>Q9Z3R4
(SEQ ID NO. 32)
MPVNLKQLAELLGLSQTTVSRALNGYPEVNAETRARVLEAVRETGYRPN
RAAQRLATGKAYSIGLVMPIAAGIDSDIHFGEFLAGLAEEAVEHDFHFV
LNPSAPEDEEATFRRLAASGNVDAVFIAYMRANDPRIEMLKALSIPFVV
HGRSIGGPRDYPFVDVDNTGAFYDAARLLIQLGHNRIALINGPEHLTFS
IRRRKGLVRALAEKGLNLDDALVHHSAMTDEHGYRSMQRFLKRPAPPTA
VLCSSTVLALGAVRAINQAGLAIGTDISLIAHDDVLPMLKPENFSVPLT
TTRSSLRAAGARIAKRLIGGILNQGDYPEQELWRAELIVRASTGPAPDR
SPLPNPSPQVGGA

Part three is a nucleotide promotor sequence which is of the desired strength in the target microorganism. In one embodiment it is independently chosen from the list of nucleotide promotor sequences comprising the nucleotide promotor sequences PcerI, PphaP, Pvan. This part determines the expression level of part two, for example the lacI gene. Exemplarily the sequence of the cerI promotor, flanked by Sall (GTCGAC) and SacI (GAGCTC) is provided as SEQ ID no. 6.

Part four is a suitable transcription terminator for termination of transcription of part two.

Part five is a nucleotide sequence for a selection marker. This may be sequence conveying an antibiotic resistance or any other type of selection. The sequence may be a sequence allowing for selection as well as having other benefits. One example of such is the publically available cloning vector pK18mob2 which provides two important properties: kanamycin resistance and the inability to replicate in hosts other than Escherichia coli and some closely related gammaproteobacteria. In one embodiment, these two features ensure that ACIT acts as a suicide plasmid (in bacteria other than E. coli and its close kin), integrating into the host chromosome. If desired, ACIT could also be used in E. coli if part five (pK18mob2) is replaced with another vector which acts as a suicide plasmid in E. coli.

Part six is a specific sequence from the host chromosome, which ensures homologous recombination between ACIT and the host chromosome and thus integration of ACIT at the chromosomally located site of a specific gene of interest.

Following, the ACIT system is presented in exemplary details, not to be understood to be limiting. In this example, the six parts are described in more detail (see FIG. 3 for a plasmid map of the parts):
1. Indigenous promoter flanked by Lac operators (120 bp, see FIG. 4 for nucleotide sequence)
2. lacI gene (1107 bp)
3. variable nucleotide promotor (variable size)
4. transcription terminator (238 bp)
5. suicide (integrating) plasmid conferring antibiotic resistance (2984 bp)
6. fragment from gene of interest for homologous recombination with chromosomal (300-1000 bp)

### Part 1. Indigenous Promoter Flanked by Lac Operators

The lac promoter from E. coli is one most widely used inducible systems in microbiology. However, in many bacteria, the lac promoter is too weak, or too leaky, or both. The solution to this problem is to insert an active promoter between two Lac operators, using the Lac promoter as a model, resp. example herein. It is important that the promotor applied is functioning within the host organism (i. e. being active) and is controlled by the two Lac operators flanking it. In one embodiment, the maximum length of the promoter, including the flanking Lac operators, is 150-200 bp. The promotor may be either a modified promotor or a native (wild-type, wt) promotor.

The two Lac operator sequences are: GGCAGTGAGCGCAACGCAATT (SEQ ID no. 1) and ATTGTGAGCGGATAACAATT (SEQ ID no. 2). These are indicated in FIG. 4. According to the Lac model as it occurs in the E. coli genome, the number of nucleotides between these two operators is optimally 71-72 bp. This number may be increased or decreased, resulting in variations in promoter performance. Within the—for example—71-72 bp should be the DNA sequence of a promoter, whose activity is preferably constitutive and strong. In one of the exemplary versions of ACIT, the promoter is from the 16S gene (RSP_4352) of Rhodobacter sphaeroides, previously shown to be a strong promoter whose activity correlates well with bacterial growth. Any other promotor can be used without leaving the scope of the invention, provided the promotor is flanked by Lac operators. Exemplarily, the promoter is flanked by the Lac operator sites given above. The 16S promoter (or any other promotor applied within ACIT) shows no activity when the Lac operators are bound to LacI. The lack of activity is because of the interaction between LacI and the two Lac operators, forming a loop in the DNA which excludes any interaction between the RNA polymerase and the promoter, thereby repressing transcription.

In the absence of LacI or when LacI is sequestered by IPTG, the DNA is free from structural hindrance to the RNA polymerase, and the promoter shows typical strong and constitutive activity. It is obvious to the person of ordinary skill in the art that the exemplary usage of the lac promoter from E. coli is not meant to confine the scope of the invention; any other promoter, providing the same features, may be used instead without leaving the scope of the invention.

Examples of further experimentally tested promotors and microorganisms are shown in FIGS. 10-13, proving the broad scope of the invention. The experiments with E. coli (FIG. 12, FIG. 13) show by example that ACIT is also working in gammaproteobacteria (for example for producing PHB within E. coli). Thus, the scope of the invention comprises gram-negative bacteria, preferably alphaproteobacteria as well as gammaproteobacteria. The promotor sequences being applied as examples are (cf. FIG. 10):
promotor sequence PcerI from R. sphaeroides:

(SEQ ID NO. 21)
GCCAAAGGAAGCCTTCCGGCTACGGGATTCCCCTCATAGCCGAAACG
GGTCGGGATGCGCAACTATAGCCGTA

promotor sequence P16S from R. sphaeroides:

(SEQ ID NO. 22)
GCGACATGAAATTGTTACGGAGCCCAAAAAATCCGCTTGCGCCCGGG
GCCGTCTGCTCCTAGAAACCGCTTCA

promotor sequence PmraZ from R. sphaeroides:

(SEQ ID NO. 23)
TCCCGCGTTTTGGGGGGCCTGGGCCCATTCTGCCCATTGCGTCCCAT
GAAATCCCATGGCATCCCTAGCTCA

promotor sequence PphaP from R. sphaeroides:

(SEQ ID NO. 24)
CAGGTGCGAAAAAATGCTGCGGTGCAGAATGAGCGGTTGATTTTCTGC
GGCGCAGCATTTATATCCTTGTCA

promotor sequence PmucR from S. meliloti:

(SEQ ID NO. 25)
AAGTTGCTGCATGCCGTTAAATTTATGTTTGGACTTTTGGCGGATCAGT
GTTAATGCAGTAAATGCAAAGTG

promotor sequence PsinI from S. meliloti:

(SEQ ID NO. 26)
GGAAAAAATGAGGAAATAAACTGTCACTATAGACAGTTACATGTGTCAT
CCGAGCCTGACAGCATCGCTACA

promotor sequence PphrR from S. meliloti:

(SEQ ID NO. 27)
AGCAATATCGCGCTTAGGTTTTAATATTAGGAAGAATTGACGTAAAACC
CCTAAAATTGCATAGTGGATTTCG

promotor sequence PlacZ from E. coli:

(SEQ ID NO. 28)
TAATGTGAGTTAGCTCACTCATTAGGCACCCCAGGCTTTACACTTTATG
CTTCCGGCTCGTATGTTGTGTGGA

promotor sequence PtrpL from E. coli:

(SEQ ID NO. 29)
TCATAACGGTTCTGGCAAATATTCTGAAATGAGCTGTTGACAATTAATC
ATCGAACTAGTTAACTAGTACGCA

promotor sequence PT5, originating from E. coli, being modified by Qiagen, thus being an artificial sequence:

(SEQ ID NO. 30)
TTTCGTCTTCACCTCGAGAAATCATAAAAAATTTATTTGCTTTGTGAGC
GGATAACAATTATAATAGATTCA

promotor sequence PeilR, originating from Enterobacter lignolyticus, being modified by Jungle Express:

(SEQ ID NO. 31)
GTCGTTGCTGCATAACAAAATTTATCAAAAAGAGTATTGACTTGGACAC
GTGTCCAACTGATACTTACAGCCA

Part 1 can be summarized as being a first DNA sequence comprising a promoter DNA sequence, being flanked by a first Lac operator DNA and a second Lac operator DNA sequence.

In one embodiment of part 1 the first lac operator sequence with at least 30, 35, 40, 45, 50, 55, 60, 65, 70, 75, 80, 85, 90, 91, 92, 93, 94, 95, 96, 97, 98 or 99% nucleotide identity to SEQ ID no. 1 is to be used. In another embodiment of part 1 a second lac operator sequence with at least 30, 35, 40, 45, 50, 55, 60, 65, 70, 75, 80, 85, 90, 91, 92, 93, 94, 95, 96, 97, 98 or 99% nucleotide identity to SEQ ID no. 2 is to be used.

In another embodiment of part 1 a promoter DNA sequence is to be used which has at least 50, 55, 60, 65, 70, 75, 80, 85, 90, 91, 92, 93, 94, 95, 96, 97, 98 or 99% nucleotide identity to SEQ ID no. 4 or SEQ ID no. 21 or SEQ ID no. 22 or SEQ ID no. 23 or SEQ ID no. 24 or SEQ ID no. 25 or SEQ ID no. 26 or SEQ ID no. 27 or SEQ ID no. 28 or SEQ ID no. 29 or SEQ ID no. 30.

The different embodiments of part 1 may be independently combined with embodiments of the other parts of ACIT.

Part 2. lacI Gene

The lacI gene exemplarily used herein is derived from E. coli and codes for the LacI repressor, a DNA-binding protein that inhibits transcription initiation. The protein contains a helix-turn-helix domain, which specifically binds to an 18-22 bp DNA sequence known as the Lac operator. Strength of transcription repression by LacI depends upon the interaction between the protein and the DNA, and this is in turn influenced by the abundance of LacI and the presence of lactose or IPTG. Lactose and IPTG disrupt the binding between LacI and its operator. It is obvious to the person of ordinary skill in the art that the exemplary usage of the lacI gene from E. coli is not meant to confine the scope of the invention; any other gene coding for a DNA-binding protein, providing the same features, may be used instead without leaving the scope of the invention.

Part 2 can be summarized as being a second DNA sequence encoding for a repressor to bind the lac operators of i).

In one embodiment of part 2 the second DNA sequence encoding for a repressor to bind the lac operators of i) is a DNA sequence with at least 50, 55, 60, 65, 70, 75, 80, 85, 90, 91, 92, 93, 94, 95, 96, 97, 98 or 99% nucleotide identity to SEQ ID no. 8. In another embodiment of part 2 the second DNA sequence encoding for a repressor to bind the lac operators of i) is a DNA sequence with at least 50, 55, 60, 65, 70, 75, 80, 85, 90, 91, 92, 93, 94, 95, 96, 97, 98 or 99% nucleotide identity to SEQ ID no. 33. The different embodiments of part 2 may be independently combined with embodiments of the other parts of ACIT.

### Part 3. Nucleotide Promotor

FIG. 1 schematically shows that within ACIT several promotor systems may be applied, indicating the great variability/applicability of the ACIT system—here exemplarily shown by either the promoter for cerI or the promoter for phaP.

The promotors exemplarily shown in FIG. 1 and in FIG. 10 are not meant to be restrictive to the scope of the invention. Any other promotor may be used, e.g. Pvan (VanR), where vanilic acid is the compound used for induction. Indeed, the possibility to use different promotors within ACIT is one of the core features of ACIT, since part of the design of ACIT is the use of specific DNA restriction digest sites (see FIG. 3 for locations of the DNA restriction sites), resp. specific DNA restriction cleavage sites, which allow the replacement of any part without disturbing any of the other parts. The expressions “digest sites” and “cleavage sites” are used herein synonymously indicating sites of precise cleavage of the DNA sequence without resulting in further decay of the DNA sequence. Hence, in each case, replacing a complete promotor sequence can be performed in a one-step cloning process.

Promoter of cerI (PcerI)

The first promoter exemplarily described herein is the cerI promoter, referred to as PcerI. In its native location in the chromosome of Rhodobacter sphaeroides, it controls the expression of Cerl, the synthase for a quorum sensing autoinducer molecule. This promoter has been shown to be dependent upon the presence of autoinducers (positive autoregulation). The two important features of this promoter for its use in ACIT are its high activity and that this activity occurs very early in the growth phase. These two conditions mean that the promoter of cerI is suited for the early, strong expression of LacI, which in turn, provides a nice control over the modified promoter and thus the expression of the gene of interest.

Promoter of phaP (PphaP)

The second exemplary variant of the ACIT system uses the promoter of phaP. The reason that this promoter was exemplarily used is because PcerI does not function in any bacterium other than R. sphaeroides, due to its dependency upon both the autoinducer molecules and CerR, the autoinducer receptor protein. Therefore, PphaP was used in ACIT constructs designed for bacteria other than R. sphaeroides, since PphaP was found to be active in a range of different bacteria. PhaP is a phasin, a protein which binds to polyhydroxybutyrate (PHB) which accumulates as a carbon storage granule in many microbes. In these other bacteria, PphaP activity was constitutive, from early in the growth phase to stationary phase. This provided strong expression of LacI and thus good control over the expression of the gene of interest.

Various promotors can be applied as part 3 of ACIT. In one embodiment the second promoter sequence of part 3 is having a size in between 8 bp and 300 bp, preferably in between 10 bp to 250 bp and more preferably up to 200 bp. The promotor of part 3 of ACIT can be the same nucleotide promotor as is comprised by part 1 or a different one.

In one embodiment of part 3 the second nucleotide promotor sequence is a DNA sequence having at least 50, 55, 60, 65, 70, 75, 80, 85, 90, 91, 92, 93, 94, 95, 96, 97, 98 or 99% nucleotide identity with SEQ ID no. 10 or a DNA sequence having at least 50, 55, 60, 65, 70, 75, 80, 85, 90, 91, 92, 93, 94, 95, 96, 97, 98 or 99% nucleotide identity with SEQ ID no. 11.

The different embodiments of part 3 may be independently combined with embodiments of the other parts of ACIT.

### Part 4. Transcription Terminator

Upon integration of ACIT into the host chromosome, it is important that a strong transcription terminator be included to silence the native promoter controlling transcription of the gene of interest. To ensure this, ACIT carries the rpoClthrA tandem terminator, derived from E. coli. It is obvious to the person of ordinary skill in the art that the exemplary usage of the rpoClthrA tandem terminator, derived from E. coli, is not meant to confine the scope of the invention; any other terminator, providing the same features, may be used instead without leaving the scope of the invention.

Part 4 can be summarized as being a fourth DNA sequence comprising a transcription terminator sequence which is terminating the transcription of part 6.

In one embodiment of part 4 the fourth DNA sequence is a DNA sequence having at least 50, 55, 60, 65, 70, 75, 80, 85, 90, 91, 92, 93, 94, 95, 96, 97, 98 or 99% nucleotide identity with SEQ ID no. 9.

Different embodiments of part 4 may be independently combined with embodiments of the other parts of ACIT.

Part 5. Suicide Plasmid pK18mob2

The essential components of an integrating plasmid are that it carries an antibiotic resistance marker and a replicating region which allows self-replication in E. coli (to allow conjugation) but not in the bacterium of interest. The plasmid pK18mob2 has frequently been used as an integrating (also known as suicide plasmid) vector suitable for research on a wide range of bacteria, including alphaproteobacteria and Gram positive bacteria. It carries the pMB1 replicon, meaning that self-replication is restricted to E. coli and closely related species of the genera Salmonella and Serratia. Also importantly for the ACIT construct, it supplies a multiple cloning site, a lacZ alpha fragment and sites for M13 primers for sequencing. Upon insertion into the chromosome, a selection of primers can be used to confirm the insertion via PCR (see FIG. 5). Finally, pK18mob2 carries a kanamycin resistance cassette, allowing for the selection of homologous recombination events. It is obvious to the person of ordinary skill in the art that the exemplary usage of the plasmid pK18mob2 is not meant to confine the scope of the invention; any other plasmid, providing the same features, may be used instead without leaving the scope of the invention.

Part 5 can be summarized as being a fifth DNA sequence comprising a sequence for a selection marker, preferably one conferring antibiotic resistance.

In one embodiment of part 5 the fifth DNA sequence comprising a sequence for a selection marker is a DNA sequence having at least 50, 55, 60, 65, 70, 75, 80, 85, 90, 91, 92, 93, 94, 95, 96, 97, 98 or 99% nucleotide identity with SEQ ID no. 7.

Different embodiments of part 5 may be independently combined with embodiments of the other parts of ACIT.

Part 6. Fragment from Gene of Interest for Homologous Recombination with Chromosome

Integration into the chromosome simultaneously silences the indigenous regulatory control over that gene and replaces it with the inducible system. To ensure that ACIT integrates into the chromosome at a specific location (i.e., directly upstream of the gene of interest), a fragment of 300-1000 bp that is identical to the gene of interest (allowing homologous recombination) must be present within ACIT. Usually, the fragment will begin upstream of the native ribosome-binding site of the gene of interest and stop somewhere within the coding region.

The fragment of the gene of interest needs to be of a suitable length to allow homologous recombination. This may be at least 150 or 200 bp in some cases. For most bacteria, a region of at least 200, preferably at least 300 bp is sufficient to ensure homologous recombination. Typically, the fragment size is not exceeding 3000 bp or is less than 2500 bp, preferably less than 2000 bp. In one embodiment the fragment size is selected from 250 bp to 1500 bp, more preferably from around 300 bp to around 1000 bp. For selection of the fragment of the gene of interest, the fragment must include the translation start (usually ATG) preceded by a suitable ribosome binding site, either native or synthetic. Inclusion of the transcription start is usually not necessary. Gaps within the fragment of the gene of interest may be tolerated, but will cause a lower frequency of homologous recombination.

FIG. 2 provides a detailed overview of ACIT, including the location of each of the parts before and following integration into the chromosome.

Natural gene regulation can be highly complex, presenting enormous challenges to the goal of achieving an appropriate level of control over gene expression. Many genetic components function poorly or not at all when transferred to non-host bacteria, meaning that these parts need to be tested and perhaps modified or even replaced in order to achieve the necessary level of control.

Gene expression can vary greatly from gene to gene. Some genes are constitutively expressed, others are regulated according to environmental factors (e.g., nutrient availability) or synchronised according to cell division, population density and growth phase. Also varying greatly from gene to gene is the strength of expression. Examples of strong gene expression are the rrn genes, which produce the rRNA component of the ribosomes. In E. coli, their expression activity is relatively strong, peaking during early exponential growth. Thereafter, their activity drops and is virtually silent upon entry into stationary phase. Regulation of the rrn gene expression and its impact on bacterial physiology remains mostly uncharacterized in E. coli, despite more than 50 years of research. This is likely due to a number of reasons. Firstly, each bacterial genome has several copies of the rrn genes. Secondly, the rrn genes are essential, meaning that they cannot be deleted from the genome. Thirdly, there is a lack of appropriate inducible expression systems capable of manipulating rrn gene expression. Even less is known about the regulation of rrn gene expression in bacteria other than E. coli, since there are currently no options for either gene deletion or temporarily silencing their expression.

Another example is the relA gene, responsible for the production and degradation of the hunger alarmone ppGpp. Expression of the native relA is very weak in S. meliloti. The problem arises when the leaky relA expression from a fully repressed Lac system carried on a plasmid is several times stronger than the native expression levels. This setup cannot avoid an over-expression of relA. This means that using the Lac system, relA can be over-expressed, but not depleted or even expressed at normal levels. The inability to deplete relA leaves only one option open to researchers interested in understanding the role of relA in the cell. That option is to delete or disrupt the relA gene. However, such relA mutants grow poorly and are plagued with the rapid appearance of secondary mutants, meaning that the relA mutant genotype is unstable. The ability to temporarily silence relA expression via tight artificial repression provides a solution to this problem, allowing less time for problematic secondary mutations to arise.

These two examples provide a glimpse of the challenges facing researchers attempting to study the regulation of gene expression. In each case, the conventional approach of gene deletion or disruption is unsatisfactory. In contrast, the use of ACIT, which allows the option of temporary gene silencing or depletion of gene expression, would allow for further research on these two important areas of research.

A third category of genes that are difficult to artificially regulate are the essential genes, meaning that functional loss of such genes results in cell death. Hence, temporary silencing or depletion of gene expression is the only way to study such genes. The gene dnaA is essential for controlling chromosome replication in almost every bacterial species. Artificial control over dnaA expression via the use of inducers means that chromosome replication, and thus growth, can be controlled by supplying the inducers at specific concentrations.

Part 6 can be summarized as being a sixth DNA sequence comprising at least one fragment from at least one gene of interest of suitable length which is ensuring the homologous recombination with the chromosomal sequence of the cell into which the transcription control cassette is to be transformed, whereat the fragment from the at least one gene of interest is comprising the first nucleotides of the respective gene of interest, whereat the DNA sequences of any two or more of parts 1 through 6 are connected directly to each other within the complete sequence of the transcription control cassette or are separated from each other within the complete sequence of the transcription control cassette by DNA sequences of DNA restriction cleavage sites.

In one embodiment of part 6 the fragment from the at least one gene of interest is a DNA sequence having at least 50, 55, 60, 65, 70, 75, 80, 85, 90, 91, 92, 93, 94, 95, 96, 97, 98 or 99% nucleotide identity with SEQ ID no. 12, SEQ ID no. 12 being a fragment from the gene sinR, coding for the AHL synthase.

In another embodiment of part 6 the fragment from the at least one gene of interest is a DNA sequence having at least 50, 55, 60, 65, 70, 75, 80, 85, 90, 91, 92, 93, 94, 95, 96, 97, 98 or 99% nucleotide identity with SEQ ID no. 13, SEQ ID no. 13 being a fragment from the gene expR, coding for an AHL receptor protein.

In another embodiment of part 6 the fragment from the at least one gene of interest is a DNA sequence having at least 50, 55, 60, 65, 70, 75, 80, 85, 90, 91, 92, 93, 94, 95, 96, 97, 98 or 99% nucleotide identity with SEQ ID no. 14, SEQ ID no. 14 being a fragment from the gene phaC, coding for the synthase for the production of polyhydroxybutyrate.

In another embodiment of part 6 the fragment from the at least one gene of interest is a DNA sequence having at least 50, 55, 60, 65, 70, 75, 80, 85, 90, 91, 92, 93, 94, 95, 96, 97, 98 or 99% nucleotide identity with SEQ ID no. 15, SEQ ID no. 15 being a fragment from the gene mraZ, being the first gene in the dcw gene cluster, the dcw gene cluster being relevant for controlling cell division.

Different embodiments of part 6 may be independently combined with embodiments of the other parts of ACIT.

In combining parts 1 to 6 to form the transcription control cassette ACIT the order of the parts 1 to 6 is important. This is exemplarily shown in FIG. 3 where all the parts are numbered. There are three important points to be considered:
1.) Part 6 must follow part 1.
2.) Part 4 must follow part 6.
3.) Part 2 must follow part 3.

All other orderings are not important. The parts, exemplarily presented herein, are replaceable, e.g., so that for part 4 (transcription terminator), any transcription terminator can be used, or for part 3 (promoter), any promoter can be used, and for part 1, any promoter (WT or native) that carries flanking operator sites can be used, as is described above.

An embodiment of the transcription control cassette ACIT comprises a DNA sequence having at least 50, 55, 60, 65, 70, 75, 80, 85, 90, 91, 92, 93, 94, 95, 96, 97, 98 or 99% nucleotide identity with SEQ ID no. 5, SEQ ID no. 5 comprising SEQ ID no. 14.

The subject matter of the invention comprises a vector comprising the transcription control cassette ACIT.

The subject matter of the invention comprises the usage of the transcription control cassette ACIT for the homologous or heterologous expression of at least one gene of interest in cells by transforming the transcription control cassette into these cells and cultivating them.

An embodiment of the usage of the transcription control cassette ACIT is characterised in that the at least one gene of interest is encoding an enzyme for the production of a fine chemical.

A certain embodiment of the usage of ACIT is directed at the production of PHB. This embodiment is applying the gene of interest having at least 50, 55, 60, 65, 70, 75, 80, 85, 90, 91, 92, 93, 94, 95, 96, 97, 98 or 99% nucleotide identity with SEQ

ID no. 16.

A certain embodiment of the usage of ACIT is directed at the production of curdlan. This embodiment is applying the gene of interest having at least 50, 55, 60, 65, 70, 75, 80, 85, 90, 91, 92, 93, 94, 95, 96, 97, 98 or 99% nucleotide identity with SEQ ID no. 17 and the gene having at least 50, 55, 60, 65, 70, 75, 80, 85, 90, 91, 92, 93, 94, 95, 96, 97, 98 or 99% nucleotide identity with SEQ ID no. 18 and the gene having at least 50, 55, 60, 65, 70, 75, 80, 85, 90, 91, 92, 93, 94, 95, 96, 97, 98 or 99% nucleotide identity with SEQ ID no. 19.

A certain embodiment of the usage of ACIT is directed at the production of galactoglucan. This embodiment is applying the gene of interest having at least 50, 55, 60, 65, 70, 75, 80, 85, 90, 91, 92, 93, 94, 95, 96, 97, 98 or 99% nucleotide identity with SEQ ID no. 20 and the other genes being located on the genomic map of Sinorhizobium meliloti from position 968095 until position 997228 of the megaplasmid pSymB, which are all together necessary for the production of galactoglucan.

The subject matter of the invention comprises a method for transformation of a bacterial cell with a transcription control cassette ACIT by performing transformation by any suitable transformation methods, for example but not limited to electroporation or by performing a biparental conjugation between a donator bacterium and the host bacterium.

In one embodiment, performing a biparental conjugation between a donator bacterium and the host bacterium includes the following steps:
a) providing of a transcription control cassette according to any of the preceding claims 1 to 8;
b) transformation of the donator bacterium with the transcription control cassette;
c) conjugation of the transformed donator bacterium of step b) with cells of at least one host bacterium with a section marker different than the one in the transcription control cassette;
d) selection of the host cells that have been transformed by the transcription control cassette by using the two respective selection agents;
e) isolating the transformed host cells.

### Trouble Shooting and Solutions Using the ACIT Design

Sometimes the gene of interest will be located in an operon of genes that are all co-expressed. ACIT offers a large advantage by replacing the native promoter and allowing the controlled expression of the operon. The problem arises, however, when the goal is to specifically control the expression of only one of those genes within the operon. The solution will be to delete the gene of interest, and integrate it along with ACIT at another location in the chromosome, requiring an additional step.

Sometimes the gene of interest is extremely small. A minimum of 150 bp, preferably 200 bp and more preferably 300 bp is suitable for the homologous recombination with ACIT. One solution is to use a plasmid encoded ACIT, although this can only ensure over-expression, not depletion of gene of interest. Another solution is first to delete the small gene of interest from the chromosome and then integrate it back into the chromosome under the control of ACIT.

Sometimes it is desirable to control multiple genes simultaneously at their native locations in the chromosome. Suicide integration only allows one copy of ACIT per genome. A solution is to excise ACIT (via homologous recombination, e.g., using the negative selection of sacB and a second homologous region) leaving only part 1 upstream of the gene of interest. This would mean that ACIT could then be modified at part 6 and re-integrated into the chromosome at the native site of the second gene of interest.

The experimental application of ACIT to control gene expression in several different types of microorganisms, Sinorhizobium meliloti, Agrobacterium tumefaciens, Rhodobacter capsulatus, R. sphaeroides and E. coli exemplarily revealed that with ACIT extensive control over the expression of numerous genes of interest is possible. ACIT provides tight, finely tuned control over the expression of the gene of interest, or—exemplarily—in the case of mraZ, the gene cluster of interest. In particular, ACIT control over gene expression is very tight in S. meliloti, providing excellent, non-leaky control over sinR and expR expression. Moreover, the modifications reveal the flexibility and ease of adaptability of ACIT. Thus, the examples shown are not confining the scope of the invention. It is especially advantageous introducing the ACIT system into microorganisms which already naturally produce the product of the gene of interest, for they are adapted to the product of the gene of interest so that its production does not negatively interfere with the viability of the microorganisms, thus providing much better yields within the fermentation process.

Exemplarily, the complete sequence of the ACIT-System is shown as SEQ ID no. 5, the fragment of the gene of interest in this example being a part of the sequence of the gene coding for PhaC, the synthase of PHB (polyhydroxybutyrate) in Rhodobacter sphaeroides.

The ACIT system can be used for the production of a fine chemical by selecting a fitting gene of interest. Fine chemicals are to be understood to be biopolymers, aroma compounds, sugars and starches, amino acids, peptides, polypeptides, vitamins or even chemical intermediates or antibodies.

Further examples of applicable fragments of genes of interest are (cf. also table 1, below):
Fragments of the sequence of SinR, a transcription activator which is essential for the activity of the promoter of sinI, the gene coding for the AHL (quorum sensing inducer) synthase of Sinorhizobium meliloti (e.g. SEQ ID no. 12),
Fragments of the sequence of ExpR, an AHL receptor protein of Sinorhizobium meliloti, which controls transcription in response to binding of AHLs (e. g. SEQ ID no. 13),
Fragments of the sequence of PhaC, the synthase of PHB (polyhydroxybutyrate) in Rhodobacter sphaeroides (e.g. SEQ ID no. 14),
Fragments of the sequence of mraZ, the first gene in the dcw gene cluster, which controls cell division in Rhodobacter sphaeroides (e.g. SEQ ID no. 15),

It is obvious to the person skilled in the art that ACIT can be used not only for the examples described herein (PHB etc.), but also for the fermentative production of numerous other products, e.g. terpenes like flavouring compounds, specialized proteins like human milk proteins, carbohydrate proteins, biogas, etc. This list of possible products is not to be understood as being limiting the scope of the invention.

Whenever hereinafter a range of values, for example regarding numbers of base pairs of nucleotides (bp), times etc. is mentioned, always additional sub-ranges within the mentioned ranges are contemplated and are within the present disclosure, even if not explicitly mentioned, as is known to and recognized by the person of ordinary skill in the art.

### DETAILED EMBODIMENTS OF THE INVENTION

For exemplarily testing the inventive method of genetic modification (ACIT), three bacteria were used: R. sphaeroides, E. coli and S. meliloti. In R. sphaeroides, two genes were selected as genes of interest: phaC (RSP_0832), which is the synthase for polyhydroxybutyrate (PHB), and mraZ and the dcw gene cluster that control cell division. In the case of phaC, the removal of phaC from its promoter (i.e., promoterless phaC) is known to almost completely remove PHB production. In the case of the dcw gene cluster, the promoter of mraZ is according to the state of the art considered to control the expression of mraZ as well as the entire dcw gene cluster. Upon insertion of the exemplary nucleic acid sequences derived from the genes of interest (phaC and mraZ) into ACIT, forming the inventive method of genetic modification, and the integration of ACIT at each of the two chromosomal locations, phaC and dcw expression were under the control of the promoter of the inventive method of genetic modification and IPTG levels.

Two additional genes were also selected for exemplarily testing the inventive method of genetic modification in S. meliloti: sinR and expR. SinR is the major transcription activator for the expression of SinI, the acyl homoserine lactone (AHL) synthase. ExpR is, like SinR, a transcription regulator. Unlike SinR, however, ExpR functions as the major AHL receptor, and the ExpR-AHL complex is the major activator of the wge genes that are essential for production of the exopolysaccharide galactoglucan. Importantly, the ACIT control over the expression of all four genes, phaC, mraZ, sinR and expR is measureable. Below, results are provided which demonstrate ACIT control in terms of either downstream gene expression (e.g., a SinI::mCherry fusion provides an indication of sinR expression) or downstream secondary metabolites (e.g., PHB for phaC or galactoglucan for expR) or a growth phenotype (e.g., mraZ and the dcw gene cluster).

Additionally to the two examples discussed above, a third example is presented herein, the application of ACIT for the production of curdlan. Regarding the production of curdlan via biosynthesis, the expression of three genes is necessary. Their position within the genetic map of Agrobacterium tumefaciens is well known to the person skilled in the art, for it is already published (e. g. Agrobacterium sp. CGMCC 11546, chromosome 2).

TABLE 1
Summary of exemplary genes of interest
and their associated phenotypes

| Gene of interest | Bacterium | Phenotype |
|---|---|---|
| phaC | R. sphaeroides | PHB accumulation |
| mraZ | R. sphaeroides | cell shape |
| sinR | S. meliloti | expression of SinI::mCherry |
| expR | S. meliloti | expression of wgeA: cerulean/ |
| | | galactoglucan production |
| crdA, crdS, crdC | A. tumefaciens | biosynthesis of curdlan |

### Measurement of ACIT Performance

phaC

PHB is a carbon-rich storage molecule located in the bacterial cytoplasm. When cells containing PHB are stained with Nile Red, fluorescence can be detected. The gene phaC codes for the PHB synthase. In the presence of IPTG, phaC expression results in PHB production, while in the absence of IPTG, a lack or very weak phaC expression results in very low levels of PHB production. FIG. 6A-C show exemplary results of ACIT used to control expression of phaC and PHB accumulation in R. sphaeroides. Fluorescence detected from the complex between PHB and Nile Red was detected using a fluorescence reader (FIG. 6B) and fluorescence microscopy (FIG. 6C). Cells with PHB granules appear brighter (FIG. 6C, left) than those lacking PHB granules (FIG. 6C, right).

mraZ/dcw

mraZ is the first gene of the dcw gene cluster controlling cell division and cell wall synthesis. Bacterial dcw gene clusters are highly conserved among bacteria. Their arrangement suggests that these are expressed as a single transcriptional unit. In R. sphaeroides, the dcw gene cluster consists of 20 genes. The fact that the promoter of mraZ also controls the expression of the entire dcw gene cluster means that the investigation of the importance of these genes for cell growth, and how these are regulated, is extremely difficult using conventional genetic deletion-and-complementation procedures. The large size of the cluster makes their cloning almost impossible. Furthermore, the fact that they are essential for growth means that they cannot be deleted. ACIT solves this problem by controlling their expression via the insertion of the IPTG inducible promoter in the chromosome. FIG. 7 exemplarily shows the usage of ACIT to control expression of the dcw gene cluster and cell shape in R. sphaeroides. In the absence of IPTG and dcw gene expression, cells are defective in cell division and appear up to 10 times longer than cells with IPTG (FIG. 3B).

sinR

Quorum sensing in S. meliloti depends upon the presence of acyl homoserine lactones (AHLs) as signalling molecules, which provide an indication of population density. The synthesis of AHLs depends upon SinI, the AHL synthase. The expression of SinI depends upon SinR, the major transcription regulator of sinI. Replacement of the native sinR promoter with ACIT allows for the control over downstream sinI expression. FIG. 8A-C exemplarily shows ACIT being used to control expression of sinR and the downstream control over sinI expression in S. meliloti. In the absence of IPTG, sinI expression is undetectable. Increasing levels of IPTG results in sinR expression and a corresponding increase in sin/expression.

expR

The receptor for AHLs in S. meliloti is ExpR. When the AHL-ExpR complex forms, the ExpR component binds to at least 30 different locations on the chromosome. This binding action either represses or activates the target genes. One of the targets of the AHL-ExpR complex is the wge gene cluster that is essential for the synthesis of galactoglucan. Galactoglucan is one of the major exopolysaccarides produced by S. meliloti, and is important in biofilm formation, mobility and symbiosis with legume plants. It is also strongly activated by quorum sensing through the AHL-ExpR complex. FIG. 9A-D exemplarily show ACIT being used to control expression of expR and galactoglucan production in S. meliloti. In the absence of IPTG, wge expression is undetectable. Increasing levels of IPTG results the expression of expR and a corresponding increase in wge expression.

The description of the whole wge gene cluster, necessary for the galactoglucan production via biosynthesis can be found in the publication of A. Becker et al., “The 32-kilobase exp gene cluster of Rhizobium meliloti directing the biosynthesis of galactoglucan: genetic organization and properties of the encoded gene products.”, Journal of Bacteriology, 1997, 179 (4): 1375-1384. The coordinates of the wge genes in the genomic map can be found on the NCBI web site which provides the genetic map, the wge genes are being located from position 968095 to 997228 on the megaplasmid pSymB of Sinorhizobium meliloti.

Example of Chromosomal Integration of ACIT at the Essential Gene dnaA

In order to prepare ACIT for integration into the host chromosome at the native dnaA locus, a 700 bp fragment which included the translation start plus the following 697 bp was inserted into ACIT. This fragment formed part 6 of ACIT (see FIG. 3 for location of part 6). This fragment was inserted via the restriction enzyme sites XbaI and KpnI. Sequencing of the modified ACIT (i.e., upon insertion of the fragment) was performed via the standard primer M13 (GCTGCAAGGCGATTAAGTTG, (SEQ ID no. 3)), providing a sequence of parts 1 and 6 (see FIG. 3 for location and orientation of M13 (5′->3′)).

Integration of the construct into host chromosome was performed by a simple biparental conjugation between the E. coli strain S17-1 and the bacterium of study, Sinorhizobium meliloti Rm2011. For the conjugation, S17-1 and S. meliloti were harvested from fresh agar cultures and resuspended and mixed at a ratio of 1:1. The mixture was then dropped on rich medium agar in 30 μL spots and incubated at 30° C. for 1-6 days. In some cases, depending upon the gene of interest, the presence of IPTG during conjugation may be helpful to ensure gene expression (in the case of essential genes) upon integration of ACIT into the chromosome. Following the incubation, the growth spot containing the bacterial mixture was resuspended in liquid medium and plated on complex agar supplemented with kanamycin (to select for ACIT) and spectinomycin (to select for S. meliloti). Spectinomycin is important because it prevents growth of S17-1. Alternatively, some other condition such as minimal salts agar with does not support the growth of S17-1 could be used. Single colonies should appear within a week of incubation.

Confirmation of integration of ACIT at the chromosomal site of the gene of interest was performed by using colony PCR. Ideally, for the colony PCR, one primer should be specific to ACIT and a second primer specific to the chromosome. See FIG. 5 for map of ACIT along with location of primers.

Exemplary description of the transformation of a host (a host bacterium) with ACIT:

Transformation of the host bacterium with ACIT is performed by a simple biparental conjugation between the donator bacterium, e.g. Escherichia coli strain S17-1, and the host bacterium, e.g. Sinorhizobium meliloti Rm2011. First, as a preparative step, ACIT is used to transform E. coli, either by calcium competent E. coli, or by electroporation of E. coli using a common method readily available in the published literature. Then, for conjugation, E. coli and S. meliloti are harvested from separate, fresh agar cultures and resuspended in fresh liquid medium and mixed at a ratio of 1:1. The mixture is then pipetted onto rich medium agar (with yeast extract) in 30 UL spots and incubated at 30° C. for 1-2 days. Conjugation occurs during this growth period. In some cases, depending upon the gene of interest, the presence of IPTG during conjugation may be helpful to ensure gene expression (in the case of essential genes) upon integration of ACIT into the chromosome. Following the incubation, the spot containing the bacterial mixture on agar is resuspended in 0.5 mL of liquid medium. About 30-50 UL of the resuspension is plated on growth agar supplemented with kanamycin (to select for ACIT) and spectinomycin (to select for S. meliloti). Spectinomycin is important because it prevents growth of E. coli but not S. meliloti. Alternatively, some other condition could be used such as minimal salts agar, which supports the growth of S. meliloti but not the growth of E. coli. Single colonies should appear within a week of incubation. Kanamycin (or Neomycin) is important because it allows the growth of only the transformants.

Confirmation of integration of ACIT at the chromosomal site of the gene of interest is performed by using colony PCR. Ideally, for the colony PCR, one primer should be specific to ACIT and a second primer specific to the chromosome. See FIG. 5 for a map of ACIT along with the location of primers.

Exemplary description of performing the biparental conjugation:

Step a) is a preparative step wherein the transcription control cassette ACIT is used to transform the donator bacterium, thus providing a transformed donator bacterium. Step b) is a conjugation step wherein cells of the transformed donator bacterium from step a) and cells of the host bacterium which are to be transformed with the transcription control cassette ACIT 1 are harvested from separate cultures, resuspended in fresh liquid medium, mixed—preferredly at a ratio of 1:1—, the mixture then being pipetted onto rich medium agar in spots and incubated, whereat incubation temperature is between 20° C. and 45° C. and incubation time is in between 4 hours and 3 days. Step c) is a selection step wherein a spot, being prepared and incubated according to step b), containing the bacterial mixture on agar is resuspended in an amount of liquid medium, then at least a part of this resuspension is plated on growth agar supplemented with a first antibiotic allowing selection for cells containing the transcription control cassette ACIT and a second antibiotic allowing selection for the cells of the host bacterium which are to be transformed with the transcription control cassette ACIT or at least a part of this resuspension is plated on growth agar supplemented with an antibiotic allowing selection for cells containing the transcription control cassette ACIT, whereat the growth agar provides conditions which are preventing only the donator bacteria from growing, so that cells of the host bacterium containing the transcription control cassette ACIT can grow, then the growth agar being plated with at least a part of the resuspension is incubated until single colonies of the host bacterium, being transformed with the transcription control cassette ACIT, have been grown.

### DESCRIPTION OF THE DRAWINGS

FIG. 1: FIG. 1 schematically shows the widespread applicability of ACIT and a simple overview of the ACIT layout.

FIG. 2: Scheme of the native (upper panel) and the modified chromosome after insertion of ACIT, allowing control over the expression of the chromosomal copy of a gene of interest.

FIG. 3: Map of ACIT showing the location and orientation of the parts of ACIT, including the suicide vector pK18mob2 and the M13 primer used for sequencing: sequence HindIII, flanking modified 16S promoter: AAGCTT; sequence XbaI, flanking modified 16S promoter: TCTAGA; transcription terminator (“omega”), KpnI: GGTACC; Sequence of phaP promoter, Sall: GTCGAC; Begin of lacI: SacI: GAGCTC; End of lacI: EcoRI: GAATTC.

FIG. 4: Example of nucleotide sequence and location of 16S promoter flanked by Lac operators, forming part 1 of ACIT:

(SEQ ID NO. 4)
AAGCTTGGCAGTGAGCGCAACGCAATTAAATTGTTACGGAGCCCA
AAAAATCCGCTTGCGCCCGGGGCCGTCTGCTCCTAGAAACCGCTT
CACCGAGACATTGTGAGCGGATAACAATTCTAGA

FIG. 5: Scheme of ACIT designed for controlling expression of the gene of interest dnaA. Following integration into the chromosome, PCR analysis was used to confirm the location and orientation of ACIT in the chromosome.

FIG. 6A: Scheme of the native and the modified chromosome after insertion of ACIT, allowing control over the expression of the chromosomal copy of phaC.

FIG. 6B: Detection of fluorescence in cells after staining with Nile Red. WT=wild type. phaC promoterless is a control strain which has been modified to remove the promoter of phaC from the chromosomal copy of phaC. Error bars represent standard deviation from 3 biological replicates.

FIG. 6C: Merged photograph (bright field and fluorescence, 488 ex, 585 em) of the wild type (left) and the promoterless phaC strain (right). Bright spots within cells indicate PHB granules. Bars indicate 2 μm.

FIG. 7: Part A shows a scheme of the native (upper panel) and an exemplarily modified chromosome after insertion of ACIT_mraZ, allowing control over the expression of the dcw gene cluster. Part B shows that cells appear normal during exponential growth in the presence of IPTG (left panel), but exert massive elongation in the absence of IPTG (right panel). Bars indicate 2 μm.

FIG. 8A: Scheme of the native (upper panel) and the modified chromosome after insertion of ACIT_sinR, allowing control over the expression of sinR.

FIG. 8B: Quorum sensing mildly slows growth in S. meliloti. Higher levels of IPTG result in slower growth via the expression of sinR.

FIG. 8C: Expression of sinR has a strong impact on the expression of SinI fused to mCherry. Error bars included for one curve are typical for all curves, and represent the standard deviation from 4 independent cultures; these are omitted from most curves to avoid cluttering the graphic.

FIG. 8D: Regulatory scheme outlining the role of SinR and SinI within the quorum sensing regulation of galactoglucan production.

FIG. 9A: Scheme of the native (upper panel) and the modified chromosome after insertion of ACIT_expR, allowing control over the expression of expR.

FIG. 9B: Regulatory scheme outlining the role of ExpR within the quorum sensing regulation of galactoglucan production.

FIG. 9C: Galactoglucan production (opaque formation) on a solid agar culture upon addition of IPTG (broken lined ring indicates area of IPTG addition).

FIG. 9D: IPTG-induced expression of ExpR has a strong impact on the expression of wgeA, the first gene in the wge gene cluster.

FIG. 10A: Examples of DNA sequence of promoters from various bacteria, general setup of the promoter elements with respect to the Lac operators.

FIG. 10B: Examples of DNA sequence of promoters from various bacteria, alignment of the DNA sequences of various promoters, showing the 71-72 nucleotides that are flanked by the 20 bp Lac operators. The promoter elements −35 and −10 are shaded grey. The transcription start (+1) is in bold and underlined. Note that the PT5 Lac operator was artificially inserted by Qiagen into the expression vecors pQE that use the T5 promoter. Also underlined are naturally occurring protein-DNA binding sites known to activate or repress promoter activity. (Examples of transcription activators include the LuxR-like regulators CerR for PcerI and SinR for PsinI. Examples of transcription repressors include ExpR for PphrR, LacI for PT5 and EilR for PeilR.)

FIG. 11: FIG. 11 shows that ACIT can use a promoter other than the 16S promoter. The modified 16S promoter of ACIT was replaced with the phaP promoter (PphaP) from R. sphaeroides, modified with Lac operators. Here, PphaP and IPTG were used to control expression of the gene for mVenus in S. meliloti. For this, the PphaP::mVenus fusion was carried on a low copy plasmid pPHU231. As a control, S. meliloti lacking the lacI gene (empty boxes) shows the maximal activity of PphaP (no repression by LacI). In the absence of LacI, PphaP is unaffected by the presence of IPTG. In contrast, when the lacI gene was inserted into the S. meliloti chromosome (controlled by an additional copy of the phaP promoter lacking the Lac operators), the presence of IPTG induced the expression of mVenus (filled circles). An inactive version (PphaPmut) of the phaP promoter, which lacks the −35 motif, reveals the background level of fluorescence. S. meliloti cultures were grown in a 96-well plate for 29 hours at 30° C. to stationary phase, with automatic measurements of mVenus fluorescence every hour. Each data point is the average of 8 cultures, with standard deviation indicated by error bars. IPTG was added at 0 hrs.

FIG. 12: FIG. 12 shows that ACIT can use various promoters in various bacteria. Promoters modified to include Lac operators (see FIG. 10) were used to control mVenus production in the four alphaproteobacteria, S. meliloti, A. tumefaciens, R. sphaeroides, R. capsulatus and mCherry production in E. coli. A, scheme used for regulating mVenus/mCherry production via PtrpL, P16S, PphaP and PT5. B, scheme used for regulating mVenus production via Peil. Arrow indicates activation, bar-end indicates repression. For the alphaproteobacteria, the ACIT cassette with the various promoters was carried on the broad host range vector pCV2 (pBBR). For E. coli, ACIT was carried on the high copy plasmid pK18mob2. Cultures were grown in a 96-well plate for 24 hrs at 30° C. to stationary phase. Each data point is the average of 8 cultures, with standard deviation indicated by error bars. IPTG or crystal violet (CV) was added at 0 hrs.

FIG. 13: FIG. 13 exemplarily depicts production of PHB in E. coli (a bacterium which naturally lacks the pha genes) using the phaA, phaB and phaC genes from R. sphaeroides. A, the phaABC genes were assembled into a plasmid (plasmid pK-Ptrp-phaABC) under the control of the modified trpL promoter (PtrpL) with flanking Lac operator sites. LacI was supplied from a second plasmid (pCV2), in which the expression of lac/was controlled by crystal violet (CV). Thus in the presence of IPTG, PtrpL was activated, resulting in the expression of the phaABC genes. B, measurement of Nile red fluorescence as an indicator of PHB accumulation in E. coli carrying both plasmids. Values are the averages of three independent cultures. Error bars indicate standard deviation.

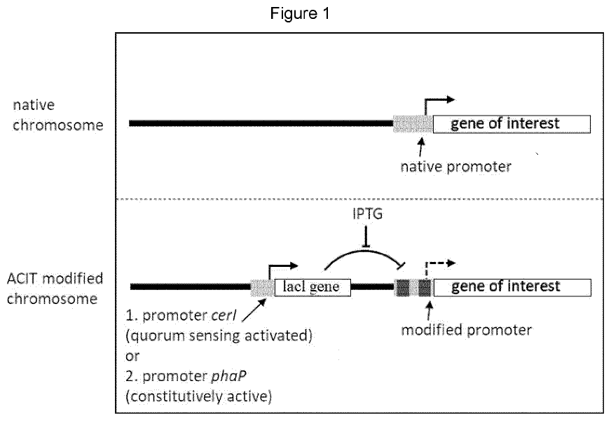

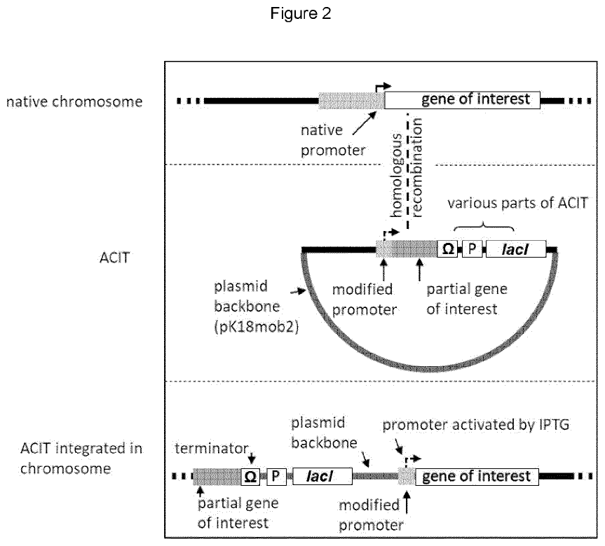

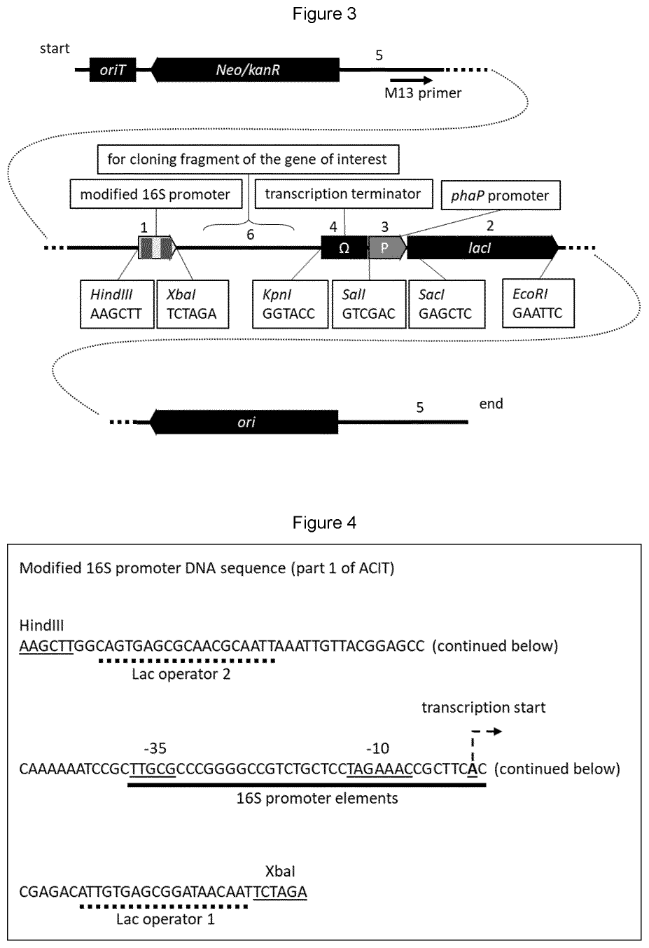

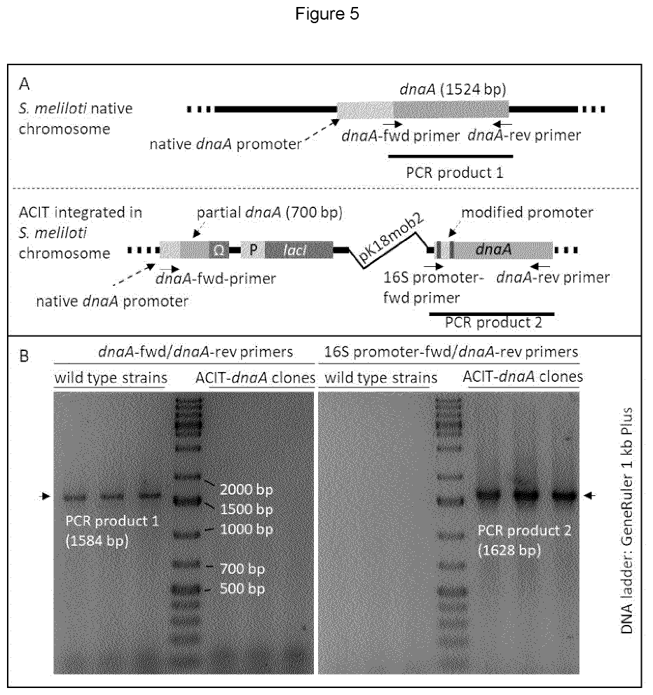

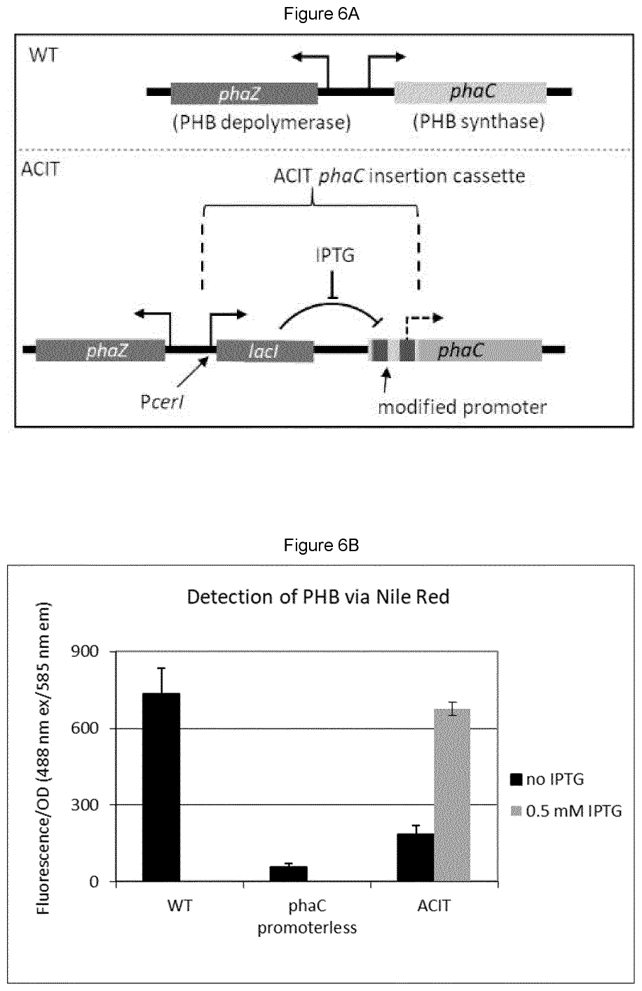

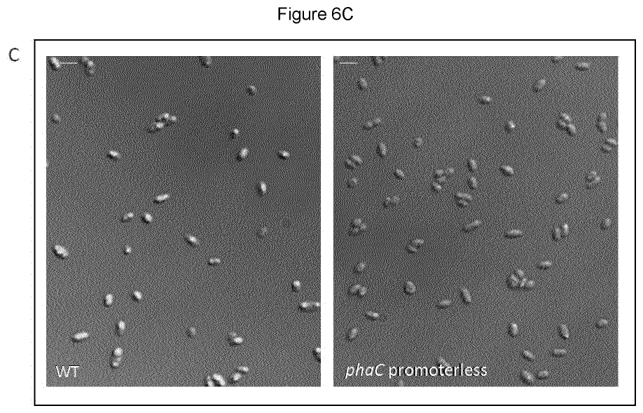

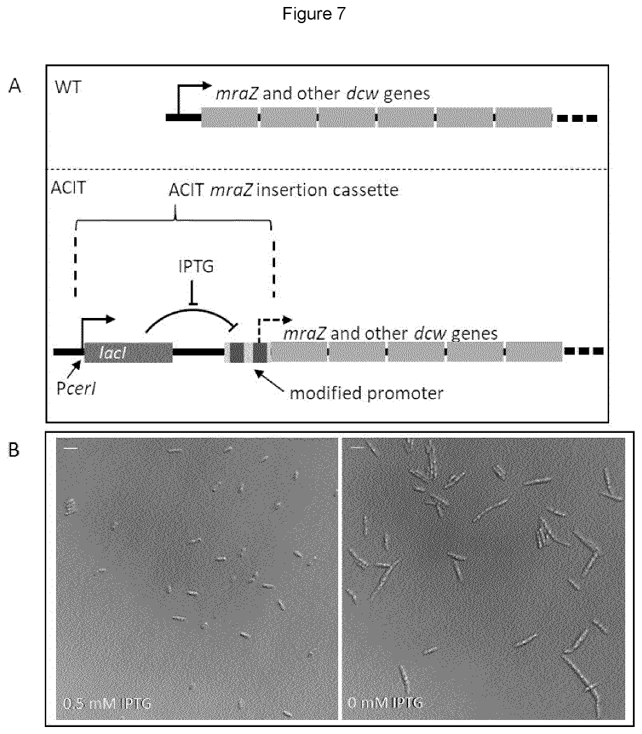

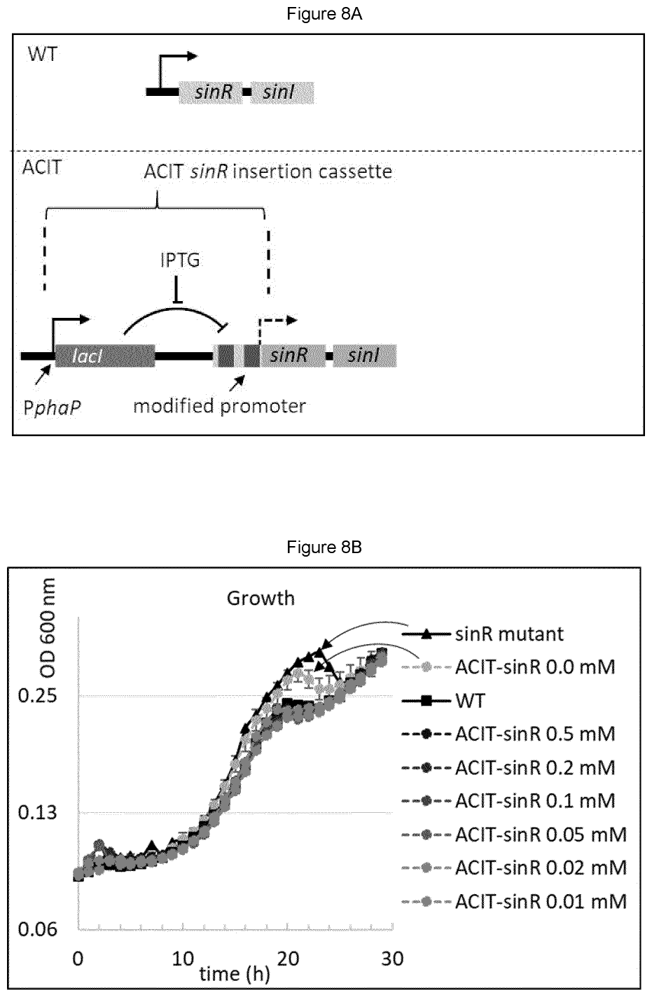

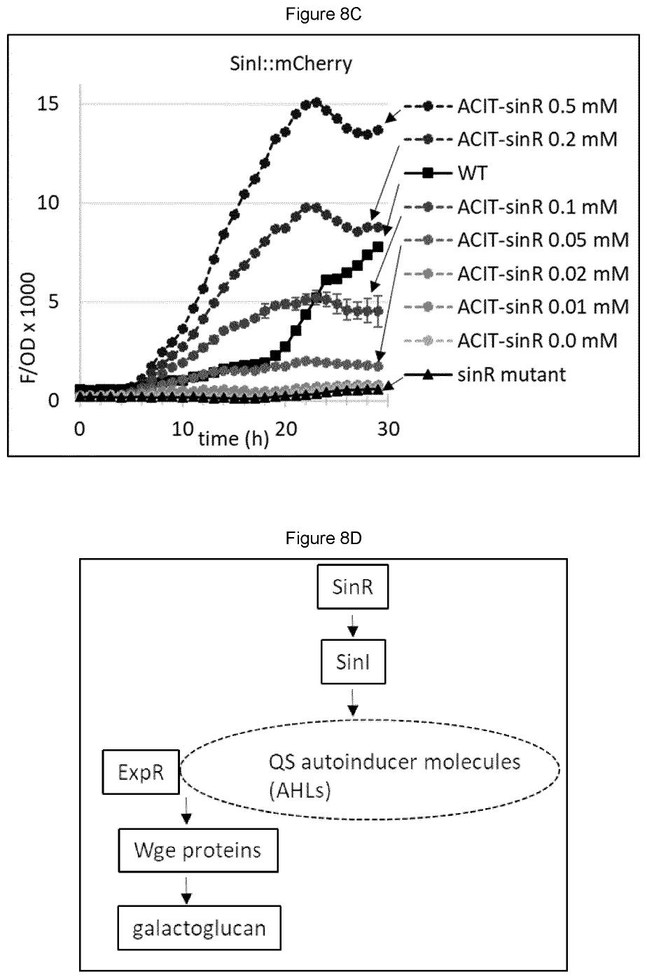

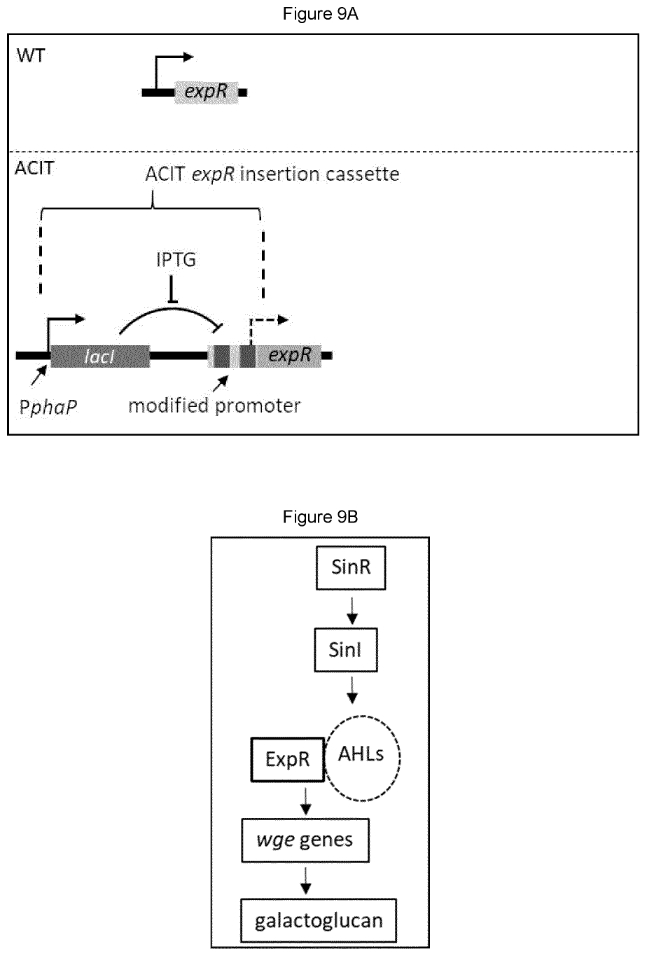

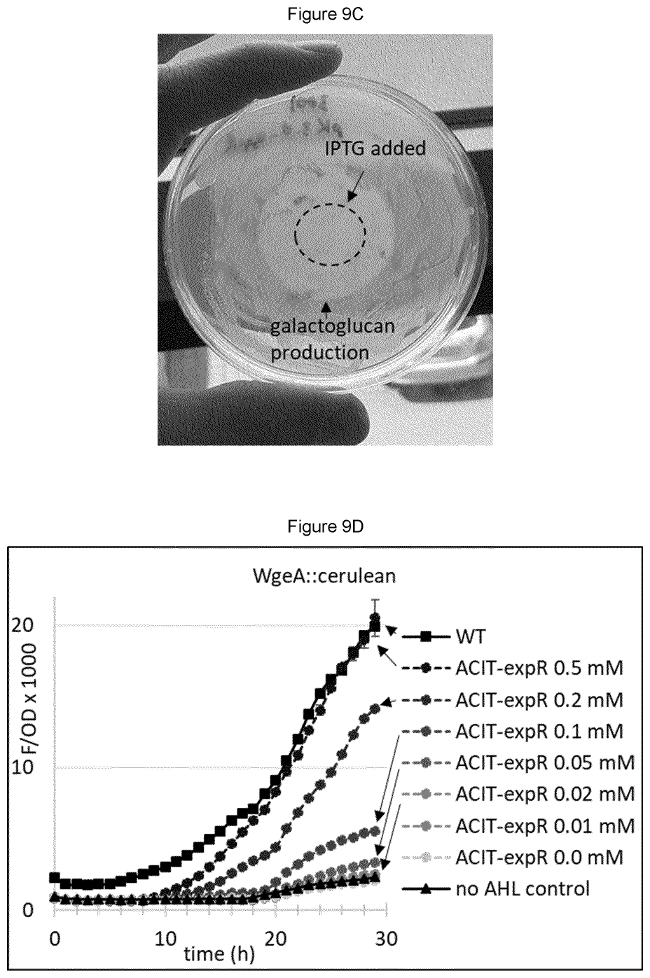

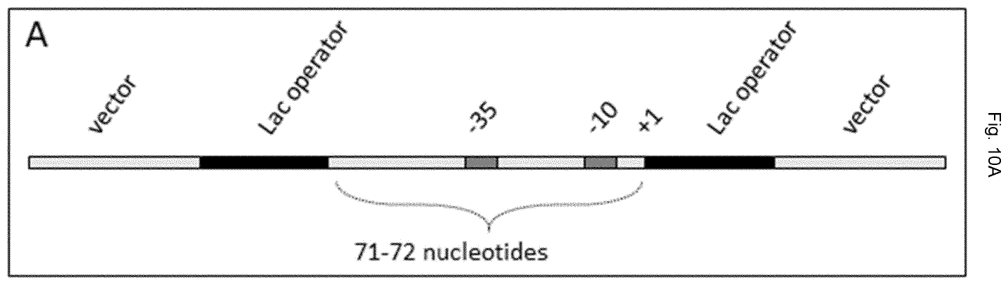

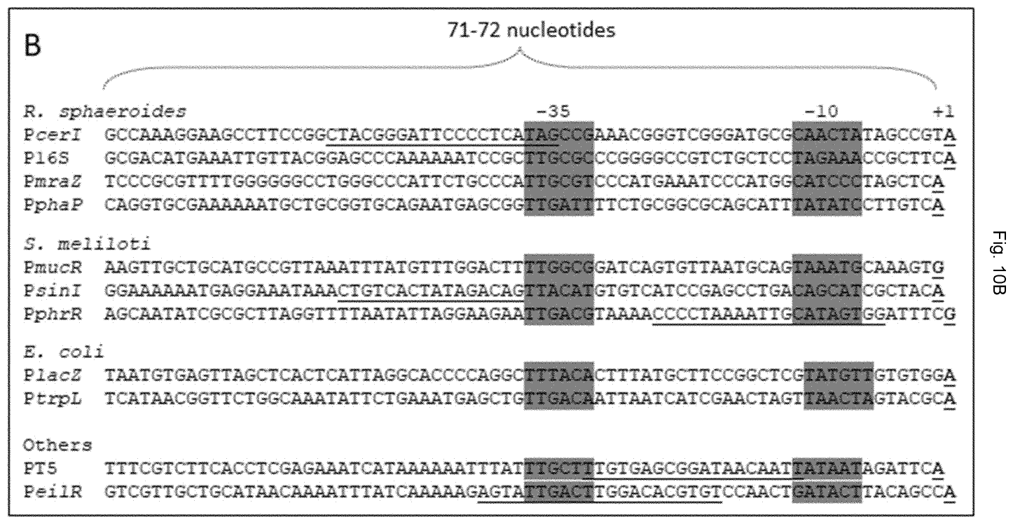

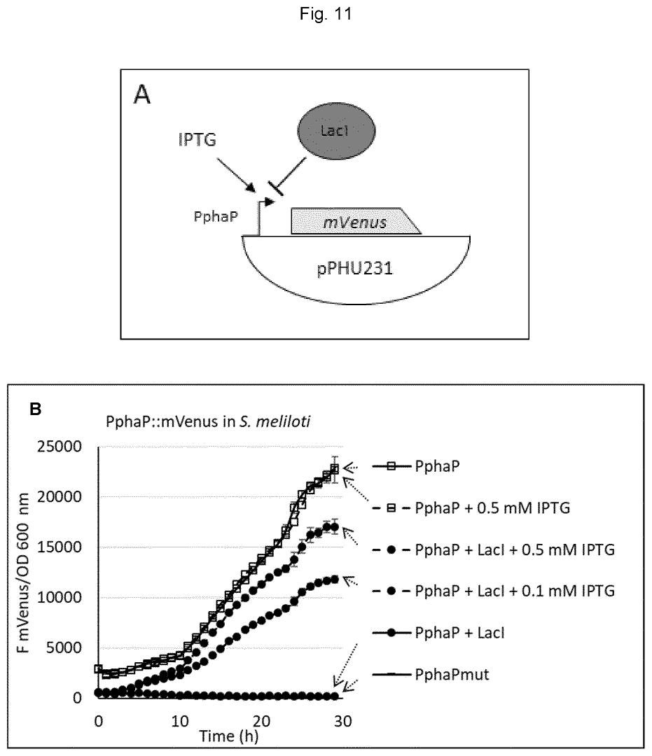

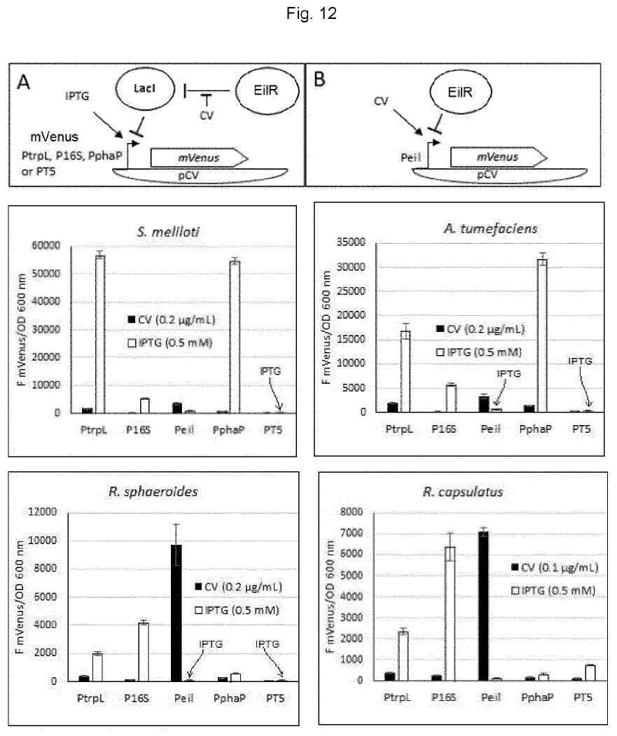

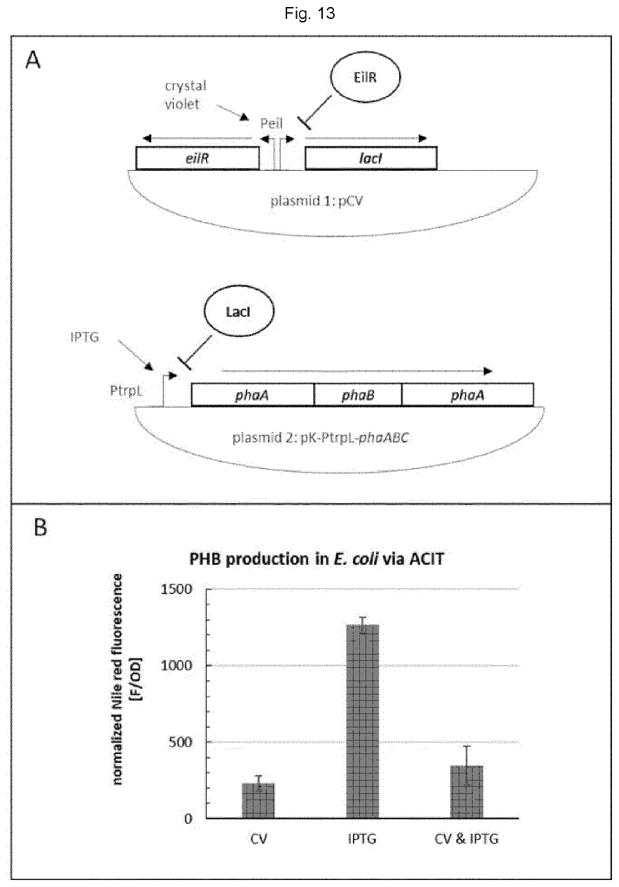

1.-15. (canceled)

16. A transcription control cassette composed for the expression of at least one gene of interest, characterized in that the transcription control cassette comprises the following parts i) through vi),
i) a first DNA sequence comprising a promoter DNA sequence, being flanked by a first Lac operator DNA sequence and a second Lac operator DNA sequence;
ii) a second DNA sequence encoding for a repressor to bind the Lac operators of i);
iii) a third DNA sequence comprising a second nucleotide promotor sequence to drive expression of ii), wherein the second nucleotide promotor is the same nucleotide promotor as is comprised by part i) or a different one;
iv) a fourth DNA sequence comprising a transcription terminator sequence which is terminating the transcription of part vi;
v) a fifth DNA sequence comprising a sequence for a selection marker;
vi) a sixth DNA sequence comprising at least one fragment from the at least one gene of interest of suitable length which is ensuring the homologous recombination with the chromosomal sequence of the cell into which the transcription control cassette is to be transformed, wherein the fragment from the at least one gene of interest is comprising the first nucleotides of the respective gene of interest;
wherein the DNA sequences of any two or more of parts i) through vi)
are connected directly to each other within the complete sequence of the transcription control cassette
or
are separated from each other within the complete sequence of the transcription control cassette by DNA sequences of DNA restriction cleavage sites.

17. A transcription control cassette according to claim 16, characterized in that the first DNA sequence comprising a promoter DNA sequence according to part i) has at least 50% nucleotide identity with a promotor DNA sequence selected from the list comprising the promotor DNA sequences SEQ ID no. 4, SEQ ID no. 21, SEQ ID no. 22, SEQ ID no. 23, SEQ ID no. 24, SEQ ID no. 25, SEQ ID no. 26, SEQ ID no. 27, SEQ ID no. 28, SEQ ID no. 29, SEQ ID no. 30.

18. A transcription control cassette according to claim 16, wherein the second nucleotide promotor sequence according to part iii) is a DNA sequence having at least 50% nucleotide identity with SEQ ID no. 10 or a DNA sequence having at least 50% nucleotide identity with SEQ ID no. 11.

19. A transcription control cassette according to claim 16, wherein the fourth nucleic acid according to part iv) is a DNA sequence having at least 50% nucleotide identity with SEQ ID no. 9
and/or
the sequence according to part v) is a DNA sequence having at least 50% nucleotide identity with SEQ ID no. 7.

20. A transcription control cassette according to claim 16, wherein the fragment from the at least one gene of interest according to part vi) is a DNA sequence having at least 50% nucleotide identity with SEQ ID no. 12, SEQ ID no. 12 being a fragment from the gene sinR, coding for the AHL synthase, or having at least 50% nucleotide identity with SEQ ID no. 13, SEQ ID no. 13 being a fragment from the gene expR, coding for an AHL receptor protein, or having at least 50% nucleotide identity with SEQ ID no. 14, SEQ ID no. 14 being a fragment from the gene phaC, coding for the synthase for the production of polyhydroxybutyrate, or having at least 50% nucleotide identity with SEQ ID no. 15, SEQ ID no. 15 being a fragment from the gene mraZ, being the first gene in the dcw gene cluster, the dcw gene cluster being relevant for controlling cell division.

21. A transcription control cassette according to claim 16, characterized in that it comprises a DNA sequence having at least 50% nucleotide identity with SEQ ID no. 5, SEQ ID no. 5 comprising SEQ ID no. 14.

22. A transcription control cassette according to claim 16, characterized in that the activity of the promoter of part i) is inducible by the presence of a compound of initialisation.

23. A transcription control cassette according to claim 22, wherein the compound of initialisation for the transcription control cassette is selected from the list comprising IPTG, allolactose, and galactose thioglycosides.

24. A vector comprising the transcription control cassette according to claim 16.

25. Usage of the transcription control cassette according to claim 16 for the homologous or heterologous expression of at least one gene of interest in cells by transforming the transcription control cassette into the cells and cultivating the cells.

26. Usage according to claim 25, characterized in that the at least one gene of interest encodes an enzyme for the production of a fine chemical.

27. A recombinant bacterial cell comprising the transcription control cassette of claim 16 and the at least one gene of interest, wherein the at least one gene of interest is operatively linked to the transcription control cassette and the transcription control cassette and the at least one gene of interest are located within the bacterial chromosome.

28. The bacterial cell according to claim 27, characterized in that it is a cell of a gram-negative bacteria.

29. The bacterial cell according to claim 27, wherein part i) is physically linked to the DNA of at least one gene of interest, optionally with DNA restriction cleavage sites in between part i) and the at least one gene of interest.

30. A method for transformation of a bacterial cell with a transcription control cassette according to claim 16 by
a) providing a transcription control cassette according to claim 16;
b) transforming a host cell with the transcription control cassette;
c) selecting the host cell that have been stably transformed with the transcription control cassette by using the respective selection agents; and
d) isolating the transformed host cell.


SEQUENCE LISTING (33 sequences)

SEQ ID NO: 1 - DNA, 21 bp, Escherichia coli
  misc_feature 1..21: note = Lac operator Sequence 1
  source 1..21: mol_type = unassigned DNA
ggcagtgagcgcaacgcaatt

SEQ ID NO: 2 - DNA, 20 bp, Escherichia coli
  misc_feature 1..20: note = Lac Operator Sequence 2
  source 1..20: mol_type = unassigned DNA
attgtgagcggataacaatt

SEQ ID NO: 3 - DNA, 20 bp, synthetic construct
  misc_feature 1..20: note = Standard sequencing primer (pUCM13-52, 5 to 3)
  source 1..20: mol_type = other DNA
gctgcaaggcgattaagttg

SEQ ID NO: 4 - DNA, 124 bp, Rhodobacter sphaeroides
  source 1..124: mol_type = unassigned DNA
aagcttggcagtgagcgcaacgcaattaaattgttacggagcccaaaaaatccgcttgcgcccggggccgtctgctcctagaaaccgcttcaccgagacattgtgagcggataacaattctaga

SEQ ID NO: 5 - DNA, 4979 bp, synthetic construct
  misc_feature 1..4979: note = Complete DNA sequence for ACIT
  source 1..4979: mol_type = other DNA
gaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggcttttccgctgcataaccctgcttcggggtcattatagcgattttttcggtatatccatcctttttcgcacgatatacaggattttgccaaagggttcgtgtagactttccttggtgtatccaacggcgtcagcggggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttatcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggcagtgagcgcaacgcaattaaattgttacggagcccaaaaaatccgcttgcgcccggggccgtctgctcctagaaaccgcttcaccgagacattgtgagcggataacaattctagacatacacaggggggacaagtcaatatggcaaccgaagagcagtctccgggttccggccgtgacgctcagttcgagcgtctgaacgcgaatctcacccgcatcgacgagctgtcgaaacggctgacggccgctctcacgaagcgcaaactgtcggaccccgcgctgcacgggccctcgggcgacgtcttcctgaaggcgatgacggcctacatggccgagatgatgcagaacccggccaagatcctcgagcatcagatcagtttctggggcaagagcctgaaacattacgtcgaggctcagcaccagctggtgaagggcgagctgaagccgccgccggacgtgacgccgaaggaccgccgcttctcgaacccgctctggcagacgcatcccttcttcaactatctcggtaccatgctgcagcgcgtacaggaaacacagaaaaaagcccgcacctgacagtgcgggctttttttttcgaccaaaggtaacgaggtaacaaccatgcgagtgttgaagtggcagaactgctgaacgcaggtctgggcggttctgataacgagtaatcgttaatccgcaaataacgtaaaaacccgcttcggcgggtttttttatggggggagtttagggaaagtcgacgcaacgcaaggcgtttcgggccaggtgcgaaaaaatgctgcggtgcagaatgagcggttgattttctgcggcgcagcatttatatccttgtcacaggataaccgggcatgagcccgctcgcaggagagagctcaggagacaaggcaatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagaattc

SEQ ID NO: 6 - DNA, 120 bp, synthetic construct
  misc_feature 1..120: note = sequence of the cerI promoter
  source 1..120: mol_type = other DNA
gtcgaccctgccccagaggaccgaaattcaaccggaaattgccaaaggaagccttccggctacgggattcccctcatagccgaaacgggtcgggatgcgcaactatagccgtatgagctc

SEQ ID NO: 7 - DNA, 2978 bp, synthetic construct
  misc_feature 1..2978: note = publically available cloning vector Pk18mob2
  source 1..2978: mol_type = other DNA
gtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggcttttccgctgcataaccctgcttcggggtcattatagcgattttttcggtatatccatcctttttcgcacgatatacaggattttgccaaagggttcgtgtagactttccttggtgtatccaacggcgtcagcggggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttatcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 8 - DNA, 1096 bp, Escherichia coli
  misc_feature 1..1096: note = Lacl gene
  source 1..1096: mol_type = unassigned DNA
aggagacaaggcaatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 9 - DNA, 219 bp, Escherichia coli
  misc_feature 1..219: note = tandem terminator sequence
  source 1..219: mol_type = unassigned DNA
atgctgcagcgcgtacaggaaacacagaaaaaagcccgcacctgacagtgcgggctttttttttcgaccaaaggtaacgaggtaacaaccatgcgagtgttgaagtggcagaactgctgaacgcaggtctgggcggttctgataacgagtaatcgttaatccgcaaataacgtaaaaacccgcttcggcgggtttttttatggggggagtttagggaaa

SEQ ID NO: 10 - DNA, 127 bp, Rhodobacter sphaeroides
  misc_feature 1..127: note = phaP promoter sequence
  source 1..127: mol_type = unassigned DNA
gcaacgcaaggcgtttcgggccaggtgcgaaaaaatgctgcggtgcagaatgagcggttgattttctgcggcgcagcatttatatccttgtcacaggataaccgggcatgagcccgctcgcaggaga

SEQ ID NO: 11 - DNA, 108 bp, Rhodobacter sphaeroides
  misc_feature 1..108: note = sequence for the promoter for cerI
  source 1..108: mol_type = unassigned DNA
cctgccccagaggaccgaaattcaaccggaaattgccaaaggaagccttccggctacgggattcccctcatagccgaaacgggtcgggatgcgcaactatagccgtat

SEQ ID NO: 12 - DNA, 423 bp, Sinorhizobium meliloti
  source 1..423: mol_type = unassigned DNA
ttcggtgttacggcatggctaatcaacaggctgtcctcaatttgctggatatcgtggaatatggaggttgcgcagaccccgagcgcttcttcgccctgatgcgtcgaaccttcaacatctcgcatctcctgtatctggaggcggagtctcttccggatggtttgagaatctgccgcctgcatcacactttcggcgcctacgccgccgaaatctacgccgccagggggctttacaggatcgacccgatcctcaagcttgcgctcggcggcgtcaggccggtggaatgggcgacagcgcggcgccgctttccggaatgcgagcccctcttcgaggcggccgaagaaatcgggctttccaccgaaggcgtggccttaccgctcccctcgcctgcgggccgcatggcgcttctcgcaatcggtgcaa

SEQ ID NO: 13 - DNA, 407 bp, Sinorhizobium meliloti
  source 1..407: mol_type = unassigned DNA
tcacatggcctggcgggttgcgcggcgcgtttttgatttgaggatgctctatgaatattacgttgctcgtacagtttctggcgctactggaagagatgaagacgcgcgaggagatcctgcccgagttcgagcgcctgctcgaccgctgcggtttcgacttctacggcatcgtgcgtcagccgaaaccgcatgagaatccgctgagacttcttctggccggacgctggccggagggctggccgcaaatctacatccgcaagaaatacgtcgttatcgatccgaccattcgcttcctcgggcatgcccagcgcggcttccgctggcgcgacacgctcgtggcattccgctcggacccgcaccgcaagcgcatggaaagcatgatggtcgaggcgcgcaatcatggcctc

SEQ ID NO: 14 - DNA, 405 bp, Rhodobacter sphaeroides
  source 1..405: mol_type = unassigned DNA
catacacaggggggacaagtcaatatggcaaccgaagagcagtctccgggttccggccgtgacgctcagttcgagcgtctgaacgcgaatctcacccgcatcgacgagctgtcgaaacggctgacggccgctctcacgaagcgcaaactgtcggaccccgcgctgcacgggccctcgggcgacgtcttcctgaaggcgatgacggcctacatggccgagatgatgcagaacccggccaagatcctcgagcatcagatcagtttctggggcaagagcctgaaacattacgtcgaggctcagcaccagctggtgaagggcgagctgaagccgccgccggacgtgacgccgaaggaccgccgcttctcgaacccgctctggcagacgcatcccttcttcaactatctc

SEQ ID NO: 15 - DNA, 468 bp, Rhodobacter sphaeroides
  source 1..468: mol_type = unassigned DNA
gatccgccgctttcgtttccggggtgcgtggtgcgcctcccggaacggcaccggacagtgaaggagccgcagggaagtggcggaggcgttcagaggcgaatacaaccagaaggttgacgccaaggcgcgggtgtcgatcccggcccccttccgtcgtgtcatcgaagcgggcgatcccaaattctccggcggccggtcgagcttcgtgctcgtctatggcggcgaccgctcctacgtcgaatgctacaccatttccgagatggagcggatcgaggaacggatccgcagcctgcccatgggcacgcccaagcggcgctatctcgaacgcaacatgatcaccctcgcgcttaacatggagctcgacgaggacggccggatcgtgctgccgcccaagggccgcgagaagctgggcatctcgcccgacgagctgaagggcggcaccgaagccacctttgcgggcacgctcaa

SEQ ID NO: 16 - DNA, 1806 bp, Rhodobacter sphaeroides
  source 1..1806: mol_type = unassigned DNA
atggcaaccgaagagcagtctccgggttccggccgtgacgctcagttcgagcgtctgaacgcgaatctcacccgcatcgacgagctgtcgaaacggctgacggccgctctcacgaagcgcaaactgtcggaccccgcgctgcacgggccctcgggcgacgtcttcctgaaggcgatgacggcctacatggccgagatgatgcagaacccggccaagatcctcgagcatcagatcagtttctggggcaagagcctgaaacattacgtcgaggctcagcaccagctggtgaagggcgagctgaagccgccgccggacgtgacgccgaaggaccgccgcttctcgaacccgctctggcagacgcatcccttcttcaactatctcaagcagcagtatctgatgaacgccgaggcggtgaatcaggccgtcgaggcgctggagcatatcgagccgtccgacaagaagcgggtcgaatatttctcgcgccagatcgtcgatcttttctcgcccacgaacttcttcggcaccaatcccgacgcgctcgaacgcgccatcgccaccgacggcgagagcctggtgcaggggctggagaatctcgtgcgcgacatcgaggccaacaacggcgatctgctcgtcacgctggccgaccccgaggcctttcaggtggggcagaacctcgccaccaccgaagggtcggtcgtctaccgcaaccgcatgttcgagctgatccagtacaagcccacgaccgagacggtccacgagacgccgctgctgatctttccgccctggatcaacaagttctacatcctcgacctcaagccgcagaattccctgctgaagtggctggtggatcagggcttcacggtcttcgtcgtctcgtgggtgaaccccgacaagagctatgccggcatcggcatggacgactacatccgcgaaggctacatgcgcgccatggccgaggtgcgctcgatcacccggcagaagcagatcaacgcggtaggctattgcatcgcgggcaccacgctcacgctgacgctggcgcacctgcagaaggcgggcgatccgtccgtacgctcggccaccttcttcaccacgctcaccgacttttcggacccgggtgaggtgggggtgttcctcaacgacgatttcgtcgacgggatcgagcggcaggtggcggtggacgggatcctcgacaagaccttcatgtcgcgcaccttcagctatctgcggtcgaacgacctgatctatcagccggcgatcaagagctacatgatgggcgaggcgccgccggccttcgacctgctctactggaacggagacggcaccaacctgccggcgcagatggcggtcgaatacctgcgtggcctgtgccagcaggaccggctggcgggcggcaccttcccggtgctgggctcgcccgtggggctgaaggatgtgacgcttcccgtctgcgccatcgcctgcgagaccgaccatatcgcgccgtggaaaagcagcttcaacggcttccgtcagttcggctcgaccgacaagaccttcattctctctcaatcgggccatgtggcgggcatcgtgaacccgcccagccgcaacaaatacggccattacaccaacgagggcccggccggcacgccggagtcgttccgggagggggccgagttccacgcgggctcctggtggccgcgctggggcgcctggctcgccgagcgatcgggcaagcaggtcccggcgcgccagccgggcgattcgaaacatcccgagctcgcgccggcgcccggatcctatgtggcggcggtgggcggggcttga

SEQ ID NO: 17 - DNA, 1458 bp, Agrobacterium tumefaciens
  source 1..1458: mol_type = unassigned DNA
atgctgttccgcaataagcctgacgcaaaatataaaggcgcatccgtaagattagcctcctatactgttctcggtgtggtggcgacggtgttttcttcttccgtgtttccgcctgtattcacctctcaaacgagccgtgctctgatcaatgcccccattattcccttgacgacgccgatcagcggcgttgtgaaaacattgaacgacaggggtggtgccaccatcgagaacgacaaggtggacaatacaacgctgatcggtctgaaggttcaactggcggcgctcgacaatgagatgcgccagaagaactcgatcgtttcggactatgcctcgcggatcgacgacctgaacagggatctttccagccagcaggcggctttgctgctgcggaccgagtccgatctcgaggctgcagaggcggccttgcagatggttacctattccacgcggatcgcaaaagccgaagcggctcgcaaacaaaaactcgtcgacaagggcgtttttcaaggcataagcgaagacatcgccgatacgatacaacttgaagatacaaaactgaaggcggcacgtgtcgctgtcaacaagctgaacaaggacatgaccttcgcgcggcaggggatttatattggatccgatctgcaatcgctgcagaacctccagcaggaaatccgcaccagaacggctgacctgatgcaggtgcggatgcagatgaccacacttgaaacacggagaagcgaactcgagaccctggcaacgactgaggcctcgcgcatcgacaggttgacgcatgcagatcttgccattccgccgggaacgacgctctacaagccggtggctgccaccgggcgggaagttgtcgccggtgacacgctcgcagaggcactcgattgccgcaacgcgtttgttgtcgctatcttctccgagcgccaggcgcaggcactctccgttggctcgcgtgttaaggtcgatgcggggggctgggcccaggagagcagtggcacggtggaaaggttggtaccgcgcacaactgaacgggtggatcttgattacgctgtcccatttccgccaacagagagacgcgaactttatgcgttcatccgcatggatggtaccgatcagcccgatctttcgcatcagaaatcctgctcggtcggcacatgggtcaatgtgaccctgccaaacgaatggctggacaggacgcgaaactacgttcacgaagcgtccgctgatctcctccaatatgcgaagaactccgcgaaggagattccgtctgtcacgcaggcagccatggcgctccttgaatcgtcgcggaacaggatcttcgcctatatgggaatggaacagccacgccagggacggctgccggagcttgcagcgccgaaccgtcaactggatgacacaaggcatgccgcgaacgggcccgttccccagaccaagatttcgcgaactcaatcctga

SEQ ID NO: 18 - DNA, 1965 bp, Agrobacterium tumefaciens
  source 1..1965: mol_type = unassigned DNA
atgtatttcagtgctgaaggtgacgttcagtcggtgctctatgtgaacctgacgattgcgattggggcgatcctgtttgcccttctcgctgatcccagaaagatggtcgacaggttggccttcagcatcatcatgttgctatcgcttggtgtctatatcgtatggcgggcaacggataccttgccgccgccggaactctccctcgaaacgctctggtgctacacctatttcaccttcgagctgatctcggtgctttatgccatggggtccatcctcatacttcttcgccgaaccgactggtcagccgttgccgatcagggagaggcatatcttgcaggcaacccgcatgcgccgctcgtcgatgtgtttatctgcacttacaacgagccgctgaacgttctcgaaaaatccatcatcgccgcgcaggcgatggattatcctcgactgcgcgtcttcgtctgtgacgacacacgtcgcggggaggtaagaacctattgcgaagccgtaggcgtgaactacgtcacacgtcccgacaacaagcacgccaaggcaggaaatctcaacaatgcgctgctccacaccaatgcgctggaagaggtttccgacttcatcatggtcctcgacgcggattttgccccccaggcaaacttcctgcggcgcgtgacgggtctcttttcggacccgaaggtggctgtcgtccagacgcctcaattctatttcaacagtgatccaattcagcacaatctcggtatagacaagagcttcgtggacgaccagcgggtcttcttcgacgtcttccagccggccaaggatgccgttggttgcgctttttgcgtcggcaccagcttcgtcgtacgccgcgccgcggtaaatggtattggtggtttcccttcggatgcgctttccgaagacatgctgctgacatatcgcctgatggaaaggggatatgtcacgcgctggctgaatgagaagttgagcgttggattgtcggcggaaggtgtacccgaatacatcacccagcgcacccgctggtgtctcggcacgatccagatcgggcttctgcggaccggacctctctggcgtggaaattttacgctgacgcagcggctgcactatctgcatggacttttctgctggctgtcgaagccgtttatcctgtgcctgctgcttgcgccgtccatctattggctgacgggcgtgtcggcgctgcaggtcgatgagctgatgttcatgaagctcggcctgtcatctcttgcgcttttctggacctattccacctggatatccggcaagaggacgcttcctctcttcaccgaagtcacccacgccctgaccgctgtacccattaccatcacgctttttcaggcaatccgtaaaccgttcgggcgcccgttcaaagtcaccgaaaagggaggagaccgatcccaggtccgtgtccacctcccgacggcgatttttttcgctttcgtgaccctgtcttcggccgtctccatcgtgctggctgtctatggtctggatgctccgtccgagctgtcctcgcgggactgcctcaatctgatctggtccgccgtcgcgatggttatcgcattcaccagcttcatttgctgcattgaattgccgcgtttcggcaaggaggaaatgatcggagtggattttcgcgggcagttgcggtccgcatcctcaacgagaccggtgcgtatcaccggcctctcgacggaaaacatcacactggctgcggttccgtcttccagcgatgtaaaggatgttttcgtaccggaggcggggtggatgcggatcagccctgcggagcacgcgcagaactccggaaagttcgatattcatccaagcgacgagcagcgccggtccattttgcgcctgttgtttcgcaaggctcctgaaaatgtcgcggaacagggcgacctgatgaaatccatgcggattcttctcgcacgggcattcgggtga

SEQ ID NO: 19 - DNA, 1266 bp, Agrobacterium tumefaciens
  source 1..1266: mol_type = unassigned DNA
atgagcatccgaaacatgatcgcgacgcttttgatggttttcgcgatgacgggcacggcggcggccgggaaaatagcggatcgtactggtctcgcggcaaagctgtgtgacggccttgcgggcaggatggaccaatcaaccgcaccgtttttgctccgcagcttcgagccggggccgtcacagaagccgctcgatccggcgctgtcaaatacggctttcacctatgataatgcgcttgcctcgattgcgcttttcgcctgtggaaaggatgacaaggcgctgaggatcgtcgaggctctgcgttatgcgcttcagcacgaccggacctatcgcgatgggcgggtgcggaatgcctaccgtgccggtgctgtcgatgttcatcagggctccatcctcctgccgggtttctggaacgagggccgcaaggcttggatggaggacgactatcaggtcggaagcgctaccggaaatcttgcctgggcagcgctggcgtttctcgccgcttttgatcgttcgggagataaatcctatctctccgctgcgtcgtcactcatcgagtggatcaaccagaataccgttggaacaggcggtctcggttttacgggcgggacgttcggtcaggatgctcaacctatacatctcgactggaaatcgacggagcataatcttgacgtctttgccgctgcagcctggctgagcgagatcgaccaggcgaaggactggcgatccgtgcaggaccgggcgtcgtcttttctcaatgccatgtggaacgatggtgagggtcgtttttacgtcggcagtttgcccggtggcgacacgccgaacaccagccagtccggcattgattccgaactttggccgatcatggccgtgccgaaattccgcgcgcgcgcggaccatgttctcgattgggtcgatcgtcattatgccgttgatggcggttttgatttcaacgacgaccgggacggcatatggctggaaggcacggcgcaagctgcgctggcgttcgagattgcggggcatcgcgctcgctcggacgcgcttcttgatacgattggagcggaattgtcgccggagagccttgtctatgccacgtcgggtcccgaattaacgacgggtttgaaaataggcccggattccacatccgcagatttcaaatatttccggttacctcacatcggcgcgacggcatgggggatactcgctgcgaaaggctggaatccgttcgtcaggaggggagtttcaagtatgcacgaggcagaataa

SEQ ID NO: 20 - DNA, 660 bp, Sinorhizobium meliloti
  source 1..660: mol_type = unassigned DNA
atggccactttggaaggcggcgcatacgacgacgccctgtttggctcccgctttgacgatttcatccgcgcccatgggggtgatgatttcgtcaacggaggcaatggggacgacatcatcttcggcgacgatggtgacgacttgctgttcggcggcaacggaaacgacaccgtgttcggcggcgaaggcaccgacgttctctatggcgaaaatggcaacgatatcctggtcggcggatcgggtgacgacctgatctatggcgataacggcaacgacattctccttgggggcgccggcagcgacttcctcctcggaggcagaggcaacgacgtgttgctcggcggcgccggcaacgacgtcatcgacggcggcgccgggtccgaccttcttgccggcggtcagggcagcgatatcttcgttttcgacggcggtggcggcaacgacgtcatcctcgactttacccccggcgaagacgtcctgcagatatccaagggtatcaacggcctcgacgtcaccgcgcccgaggatctcgcttcgcgtatcactcaggtcggcggcaatgtcgtcatcgacctcggccatggcgacacgctgacgctggtcaatgccgacgccgaagacattcaggcccatcccgaaaactacttcactgtccactga

SEQ ID NO: 21 - DNA, 73 bp, Rhodobacter sphaeroides
  misc_feature 1..73: note = promotor sequence Pcerl
  source 1..73: mol_type = unassigned DNA
gccaaaggaagccttccggctacgggattcccctcatagccgaaacgggtcgggatgcgcaactatagccgta

SEQ ID NO: 22 - DNA, 73 bp, Rhodobacter sphaeroides
  misc_feature 1..73: note = promotor sequence P16S
  source 1..73: mol_type = unassigned DNA
gcgacatgaaattgttacggagcccaaaaaatccgcttgcgcccggggccgtctgctcctagaaaccgcttca

SEQ ID NO: 23 - DNA, 72 bp, Rhodobacter sphaeroides
  source 1..72: mol_type = unassigned DNA
tcccgcgttttggggggcctgggcccattctgcccattgcgtcccatgaaatcccatggcatccctagctca

SEQ ID NO: 24 - DNA, 72 bp, Rhodobacter sphaeroides
  source 1..72: mol_type = unassigned DNA
caggtgcgaaaaaatgctgcggtgcagaatgagcggttgattttctgcggcgcagcatttatatccttgtca

SEQ ID NO: 25 - DNA, 72 bp, Sinorhizobium meliloti
  source 1..72: mol_type = unassigned DNA
aagttgctgcatgccgttaaatttatgtttggacttttggcggatcagtgttaatgcagtaaatgcaaagtg

SEQ ID NO: 26 - DNA, 72 bp, Sinorhizobium meliloti
  source 1..72: mol_type = unassigned DNA
ggaaaaaatgaggaaataaactgtcactatagacagttacatgtgtcatccgagcctgacagcatcgctaca

SEQ ID NO: 27 - DNA, 73 bp, Sinorhizobium meliloti
  source 1..73: mol_type = unassigned DNA
agcaatatcgcgcttaggttttaatattaggaagaattgacgtaaaacccctaaaattgcatagtggatttcg

SEQ ID NO: 28 - DNA, 73 bp, Escherichia coli
  source 1..73: mol_type = unassigned DNA
taatgtgagttagctcactcattaggcaccccaggctttacactttatgcttccggctcgtatgttgtgtgga

SEQ ID NO: 29 - DNA, 73 bp, Escherichia coli
  source 1..73: mol_type = unassigned DNA
tcataacggttctggcaaatattctgaaatgagctgttgacaattaatcatcgaactagttaactagtacgca

SEQ ID NO: 30 - DNA, 72 bp, Escherichia coli
  misc_feature 1..72: note = promotor sequence PT5
  source 1..72: mol_type = unassigned DNA
tttcgtcttcacctcgagaaatcataaaaaatttatttgctttgtgagcggataacaattataatagattca

SEQ ID NO: 31 - DNA, 73 bp, Enterobacter lignolyticus
  misc_feature 1..73: note = promotor sequence PeiIR
  source 1..73: mol_type = unassigned DNA
gtcgttgctgcataacaaaatttatcaaaaagagtattgacttggacacgtgtccaactgatacttacagcca

SEQ ID NO: 32 - protein, 356 aa, Sinorhizobium meliloti
MPVNLKQLAELLGLSQTTVSRALNGYPEVNAETRARVLEAVRETGYRPNRAAQRLATGKAYSIGLVMPIAAGIDSDIHFGEFLAGLAEEAVEHDFHFVLNPSAPEDEEATFRRLAASGNVDAVFIAYMRANDPRIEMLKALSIPFVVHGRSIGGPRDYPFVDVDNTGAFYDAARLLIQLGHNRIALINGPEHLTFSIRRRKGLVRALAEKGLNLDDALVHHSAMTDEHGYRSMQRFLKRPAPPTAVLCSSTVLALGAVRAINQAGLAIGTDISLIAHDDVLPMLKPENFSVPLTTTRSSLRAAGARIAKRLIGGILNQGDYPEQELWRAELIVRASTGPAPDRSPLPNPSPQVGGA

SEQ ID NO: 33 - DNA, 1071 bp, Sinorhizobium meliloti
  source 1..1071: mol_type = other DNA
atgccggtcaatctcaagcaattggcggaactgctcggcctgtcccagaccacggtcagccgggcgctgaacggctatccggaggtcaatgcggaaacgcgggcacgggtgctggaggcggtgcgcgagacgggatacaggcccaaccgtgccgcacagcgcctcgcgaccgggaaagcctattcgatcgggctcgtcatgccgatcgcagccggaatcgattcggacatccatttcggtgaattcctggcggggctggcggaagaagcggtggagcacgatttccacttcgtgttgaaccccagtgcgcccgaagacgaggaggcgacattccgccggcttgcagcgagcggcaatgtcgacgctgtcttcatcgcctatatgcgcgccaacgatccccgcatcgagatgctgaaggcactttccattcccttcgtggtgcatggccgctccatcggcggtccccgggactatccgttcgtcgacgtggacaataccggcgccttctacgacgccgccaggcttctcatccagctcggccacaaccgcatcgccctgatcaacgggccggaacacctcaccttctccatccgcaggaggaaggggctggtgcgcgcgctcgcggagaagggtctcaacctcgacgatgcgctcgtccatcactctgcgatgaccgacgagcatggctatcggagcatgcagcgtttcctcaaacggccggccccgccgaccgccgtcctttgttccagtacagtgctggcgctcggcgccgtacgggcgatcaaccaggccggactcgccatcggcaccgacatctcgctcatcgcccatgacgacgtgctgccgatgctgaagcccgagaacttcagcgtaccgctgacgacgacgcgctcctcgcttcgcgcggcaggtgcacgcatcgccaagcggctgatcggcggcatactcaatcagggagactatcccgagcaggagctttggcgcgccgaactgatcgtgcgtgcctcgacggggcctgcaccggacagatcacccctccccaacccctccccacaggtgggaggggcttaa